(19) United States
(12) Patent Application Publication
(10) Pub. No.: US 2026/0097137 A1
(43) Pub. Date: Apr. 9, 2026

(54) FLUORESCENT PROBE COMPOUNDS FOR TUMOR TARGETING IMAGING, AND SYNTHESIS METHOD THEREFOR

(71) Applicant: HEBEI MEDICAL UNIVERSITY, Shijiazhuang (CN)

(72) Inventors: Lei WANG, Shijiazhuang (CN); Yining ZHANG, Shijiazhuang (CN); Zijun LUO, Shijiazhuang (CN); Lixiao GUO, Shijiazhuang (CN); Yi LIU, Shijiazhuang (CN); Huicai GUO, Shijiazhuang (CN); Ran GUO, Shijiazhuang (CN); Jing WANG, Shijiazhuang (CN); Qingzhong JIA, Shijiazhuang (CN)

(73) Assignee: HEBEI MEDICAL UNIVERSITY, Shijiazhuang (CN)

(21) Appl. No.: 19/415,861

(22) Filed: Dec. 11, 2025

Related U.S. Application Data
(63) Continuation of application No. PCT/CN2024/099003, filed on Jun. 13, 2024.

(30) Foreign Application Priority Data
Jun. 14, 2023 (CN) 202310703292.4

(51) Int. Cl. A61K 49/00; A61P 35/00

(52) U.S. Cl. CPC A61K 49/0052; A61K 49/0032; A61K 49/0043; A61P 35/00

(57) ABSTRACT

The present invention relates to fluorescent probe compounds for tumor-targeted imaging, and methods of synthesis and use thereof. The compounds utilize, for the first time, an antitumor drug comprising a pyrrolo[2,3-d]pyrimidine core structure as a targeting ligand, which is conjugated to a fluorescent dye through a linker moiety. The structure of the compound is shown in Formula I. The compound demonstrates high affinity and selectivity for tumor cells expressing target receptors, enabling localization, qualitative and quantitative analysis through in vitro and in vivo tracking, receptor affinity studies, and mechanism of action investigations, thereby exhibiting high specificity, sensitivity, and visualization capability. The fluorescent probe of the present invention achieves rapid clearance from normal tissues while maintaining prolonged retention at tumor sites, thereby enabling in vivo diagnostic functionality. It demonstrates substantial clinical application potential for clinical intraoperative navigation.

### CROSS-REFERENCE TO RELATED APPLICATIONS

The application claims priority to Chinese patent application No. 202310703292.4, filed on Jun. 14, 2023, the entire contents of which are incorporated herein by reference.

### TECHNICAL FIELD

The present disclosure pertains to the field of specific molecular targeted diagnostic agents. More particularly, it relates to fluorescent probe compounds for tumor-targeted imaging, their synthesis methods.

### BACKGROUND

Cancer has long been a century-spanning challenge confronting humanity, leading to the development of various therapeutic modalities including surgical resection, chemotherapy, radiotherapy, and biological therapy. Although complete eradication of malignant tissue via surgical resection is often unattainable, approximately 50% of cancer patients undergoing resection of detectable malignant tissue exhibit no disease recurrence, potentially experiencing prolonged life expectancy or reduced cancer recurrence rates. Consequently, surgical resection remains the most prevalent and effective therapeutic intervention. The critical importance of complete malignant tissue removal underscores the significant application value of diagnostic techniques capable of ensuring precise and thorough identification of malignant lesions.

Despite recognition of the necessity for complete tumor removal and the availability of certain identification technologies for visualizing tumor masses, many malignant tissues evade detection, leading to disease recurrence and ultimately mortality. Thus, achieving more accurate and comprehensive tumor identification represents a core problem urgently requiring solution in this field. With the discovery and application of fluorescent dyes, an intraoperative navigation technique termed “fluorescence-guided surgery” has progressively gained prominence in surgical practice. This technique utilizes light of specific excitation wavelengths to induce fluorescence from either inherent tumor autofluorescence, retained fluorescent molecules, or exogenously administered fluorescent agents internalized by cells, thereby guiding surgeons in performing precise tumor resection. However, conventional fluorescent dyes inherently lack targeting specificity. They accumulate not only in tumor tissues but also in normal tissues, potentially leading to over-resection and damage to surrounding healthy structures. Therefore, a pressing need exists for developing targeted fluorescent probes.

Owing to its highly disease-restricted expression pattern, the alpha isoform of the folate receptor (FR-α) is recognized as a promising target for developing cancer-specific diagnostic and therapeutic strategies. Concurrently, recent studies report that FR-β is specifically overexpressed on tumor-associated macrophages (TAMs) and myeloid-derived suppressor cells (MDSCs), identifying it as a marker for these immunosuppressive cells, thus providing a valuable tool for precise anti-tumor immunotherapy. As folate receptors demonstrate high binding affinity for folic acid and folate conjugates (Kd≈10⁻⁹ M) and facilitate internalization via receptor-mediated endocytosis, they represent excellent targets for tumor-targeted fluorescent probes. Folate antagonists featuring a pyrrolo[2,3-d]pyrimidine core exhibit potent antitumor activity and potential selectivity for folate receptors; however, their application as targeting ligands for fluorescent probes has not been reported.

Fluorescein isothiocyanate (FITC), a derivative of fluorescein functionalized by replacing a hydrogen atom on the bottom ring of the native structure with an isothiocyanate reactive group (—N═C═S), possesses high absorption, excellent fluorescence quantum yield, and good water solubility. It is widely used in applications including flow cytometry and remains one of the most common fluorescent markers. Compared to cyanine dyes operating in the near-infrared (NIR) region, FITC exhibits shorter excitation and emission wavelengths. While suboptimal as an imaging agent for fluorescence-guided surgery due to limitations in tissue penetration, its broad applicability makes it a versatile fluorescent probe tool. For instance, EC17, a classic FR-targeted molecular fluorescent probe comprising folic acid conjugated to FITC, was among the first applied clinically and has been shown to selectively accumulate in cancers such as breast cancer, lung adenocarcinoma, and ovarian cancer. However, due to its emission in the visible wavelength range, EC17 may fail to reveal deeply buried cancerous nodules, leading to undetected occult lesions. Recent research (J. Med. Chem. 2018, 61, 9637-9646) developed a novel NIR fluorescent probe, OTL38, by conjugating folic acid as the recognition group to an NIR cyanine dye. This probe illuminates tumor tissue at significant depths while enabling fluorescent dye accumulation in tumors with high folate receptor expression (e.g., ovarian cancer, non-small cell lung cancer) and has entered preliminary clinical trials. Nonetheless, these related studies predominantly utilize traditional folic acid molecules as the targeting ligand conjugated to the fluorophore, which can inevitably reduce receptor affinity and consequently lead to undesired accumulation in normal tissues.

In view of the above limitations, the present invention applies folate antagonists possessing a pyrrolo[2,3-d]pyrimidine core structure to the design of tumor-targeted fluorescent probes. It aims to develop a series of molecular fluorescent probes capable of specifically targeting tumor tissues and enabling in vivo imaging with higher affinity, intended for application in surgical navigation.

### SUMMARY

An object of the present invention is to provide a molecular fluorescent probe compound capable of specifically targeting tumor tissues and enabling in vivo imaging with higher affinity, concurrently providing its preparation method and applications.

To achieve the aforesaid object, the technical solution adopted in the present invention is as follows:

In a first aspect, provided is a fluorescent probe compound for tumor-targeted imaging or a pharmaceutically acceptable salt thereof. Said compound innovatively employs an antitumor drug possessing a pyrrolo[2,3-d]pyrimidine core structure as a targeting ligand. This ligand is modified via a linker molecule and conjugated to a fluorescent dye. The structure of the compound is represented by Formula I:

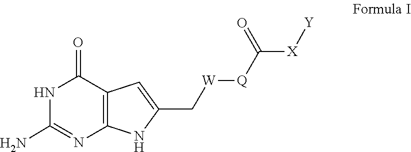

wherein W and Q are selected from the following structures:
(1) W is C(O)NH—, and Q is (CH₂)ⱼ—, wherein j is selected from 1, 2, 3, 4, or 5;
(2) W is (CH₂)ₙ—, and Q is

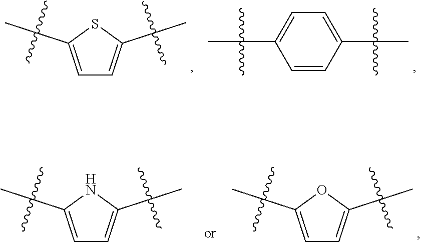

wherein n is selected from 1, 2, 3, 4, or 5;
X is an amino group or an amidation derivative group, wherein the amino group is independently selected from tyrosine, cysteine, glutamic acid, methionine, threonine, serine,

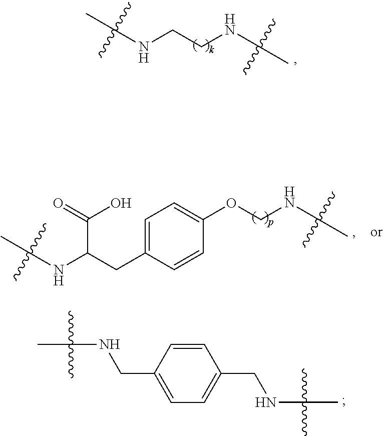

The amide derivative group represents an amidation derivative formed by the condensation of two or more structures independently selected from the following amino groups: tyrosine, cysteine, glutamic acid, methionine, threonine, serine,

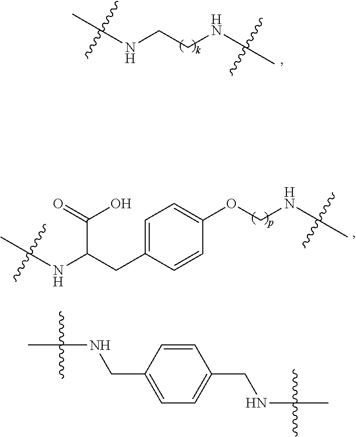

or derivatives thereof.

k is selected from 0, 1, 2, 3, 4, or 5;

p is selected from 1, 2, 3, or 4;

Y is independently selected from dyes having fluorescence excitation in the visible spectrum and emission in the near-infrared range, and the compound maintains or enhances the fluorescence of said dye.

As a further improvement of the present invention, the amide derivative group is selected from the group consisting of:

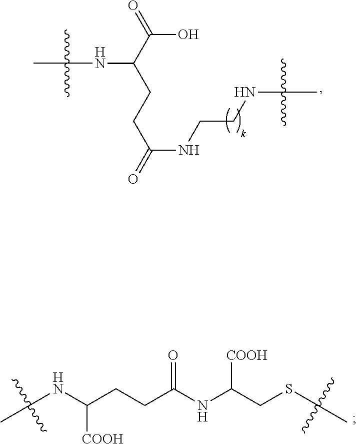

wherein k is independently selected from 0, 1, 2, 3, 4, or 5.

As a further improvement of the present invention, Y is selected from the group consisting of fluorescein isothiocyanate (FITC), dye IR-783, and S0456.

As a further improvement of the present invention, in said amino group or amide derivative group, the amino group on one side forms an amide bond with the carbonyl group linked to Q, while the amino group, thiol group, or oxy group on the other side is linked to Y.

As a further improvement of the present invention, the compound is selected from the group consisting of:

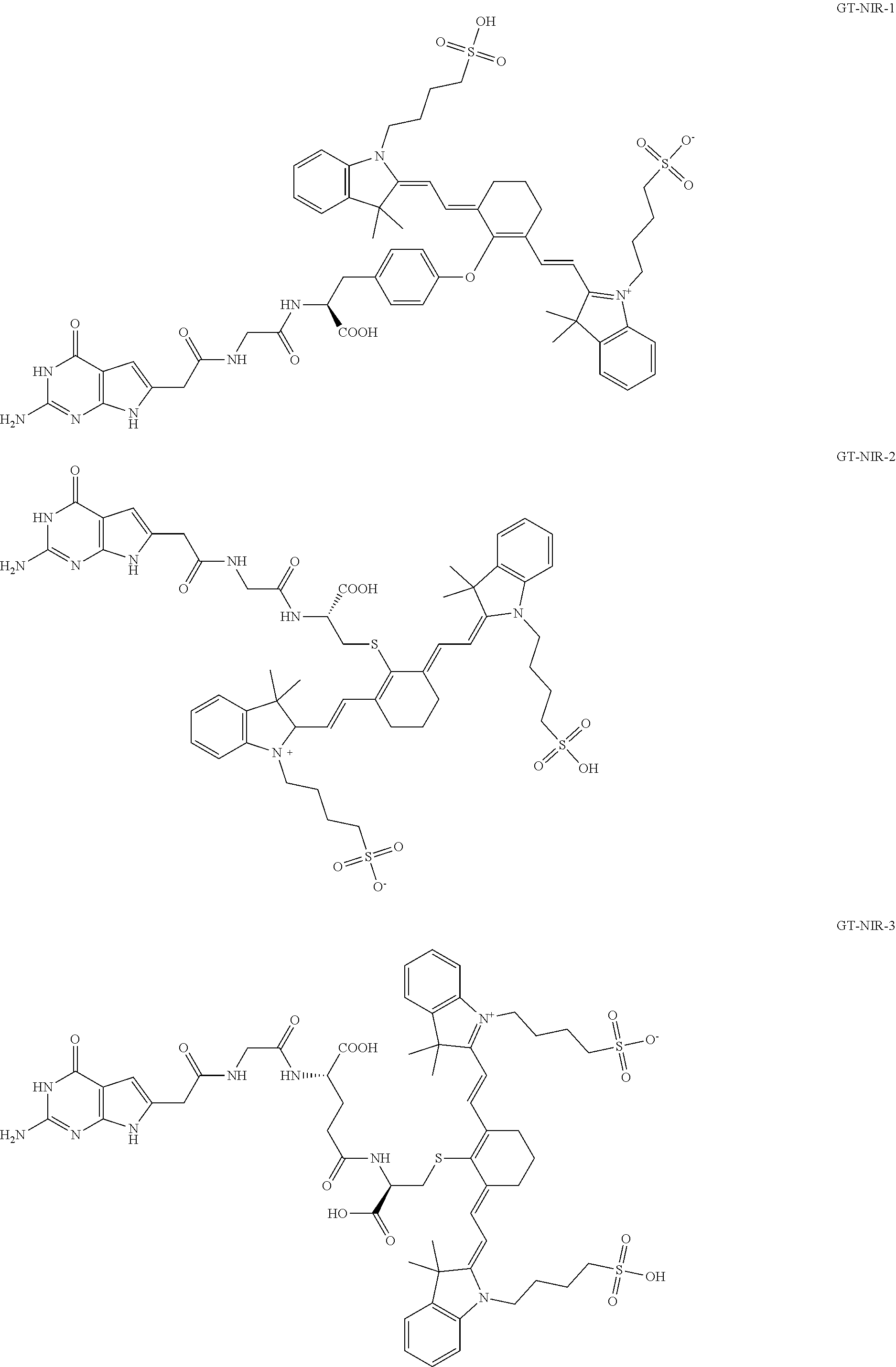 [SMILES: CC1(C)C(/C=C/C2=C(Oc3ccc(C[C@H](NC(=O)CNC(=O)Cc4cc5c(=O)[nH]c(N)nc5[nH]4)C(=O)O)cc3)C(=C/C=C3/N(CCCCS(=O)(=O)O)c4ccccc4C3(C)C)/CCC2)=[N+](CCCCS(=O)(=O)[O-])c2ccccc21 ; CC1(C)/C(=C\C=C2/CCCC(/C=C/C3N(CCCCS(=O)(=O)[O-])c4ccccc4C3(C)C)=C2SC[C@H](NC(=O)CNC(=O)Cc2cc3c(=O)[nH]c(N)nc3[nH]2)C(=O)O)N(CCCCS(=O)(=O)O)c2ccccc21 ; CC1(C)C(/C=C/C2=C(SC[C@H](NC(=O)CC[C@H](NC(=O)CNC(=O)Cc3cc4c(=O)[nH]c(N)nc4[nH]3)C(=O)O)C(=O)O)C(=C/C=C3/N(CCCCS(=O)(=O)O)c4ccccc4C3(C)C)/CCC2)=[N+](CCCCS(=O)(=O)[O-])c2ccccc21]

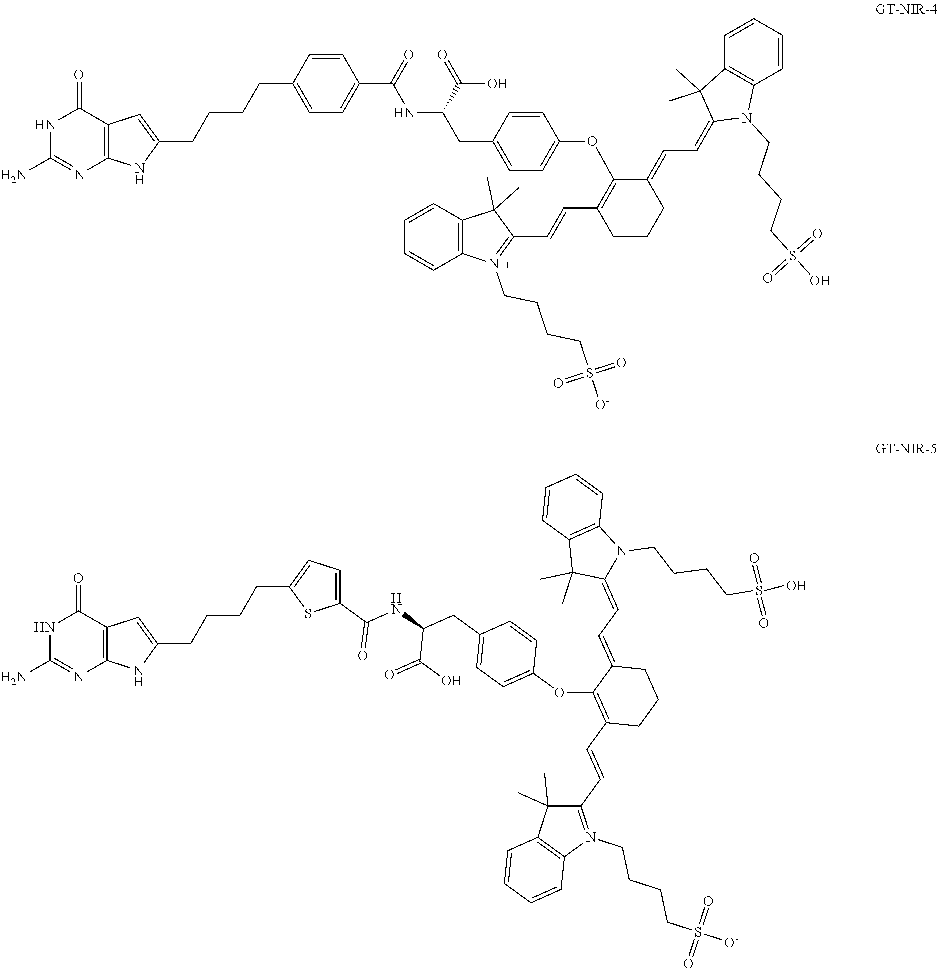

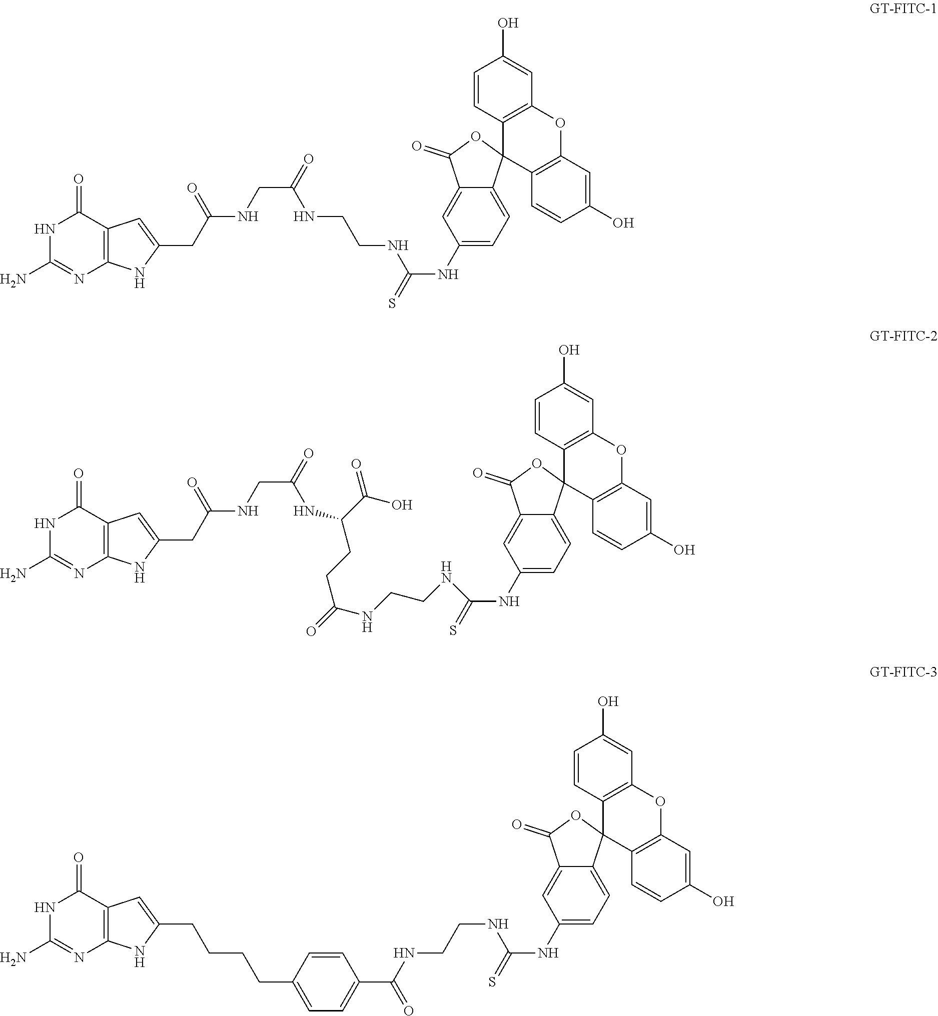 [SMILES: Nc1nc2[nH]c(CC(=O)NCC(=O)NCCNC(=S)Nc3ccc4c(c3)C(=O)OC43c4ccc(O)cc4Oc4cc(O)ccc43)cc2c(=O)[nH]1 ; Nc1nc2[nH]c(CC(=O)NCC(=O)N[C@@H](CCC(=O)NCCNC(=S)Nc3ccc4c(c3)C(=O)OC43c4ccc(O)cc4Oc4cc(O)ccc43)C(=O)O)cc2c(=O)[nH]1 ; Nc1nc2[nH]c(CCCCc3ccc(C(=O)NCCNC(=S)Nc4ccc5c(c4)C(=O)OC54c5ccc(O)cc5Oc5cc(O)ccc54)cc3)cc2c(=O)[nH]1]

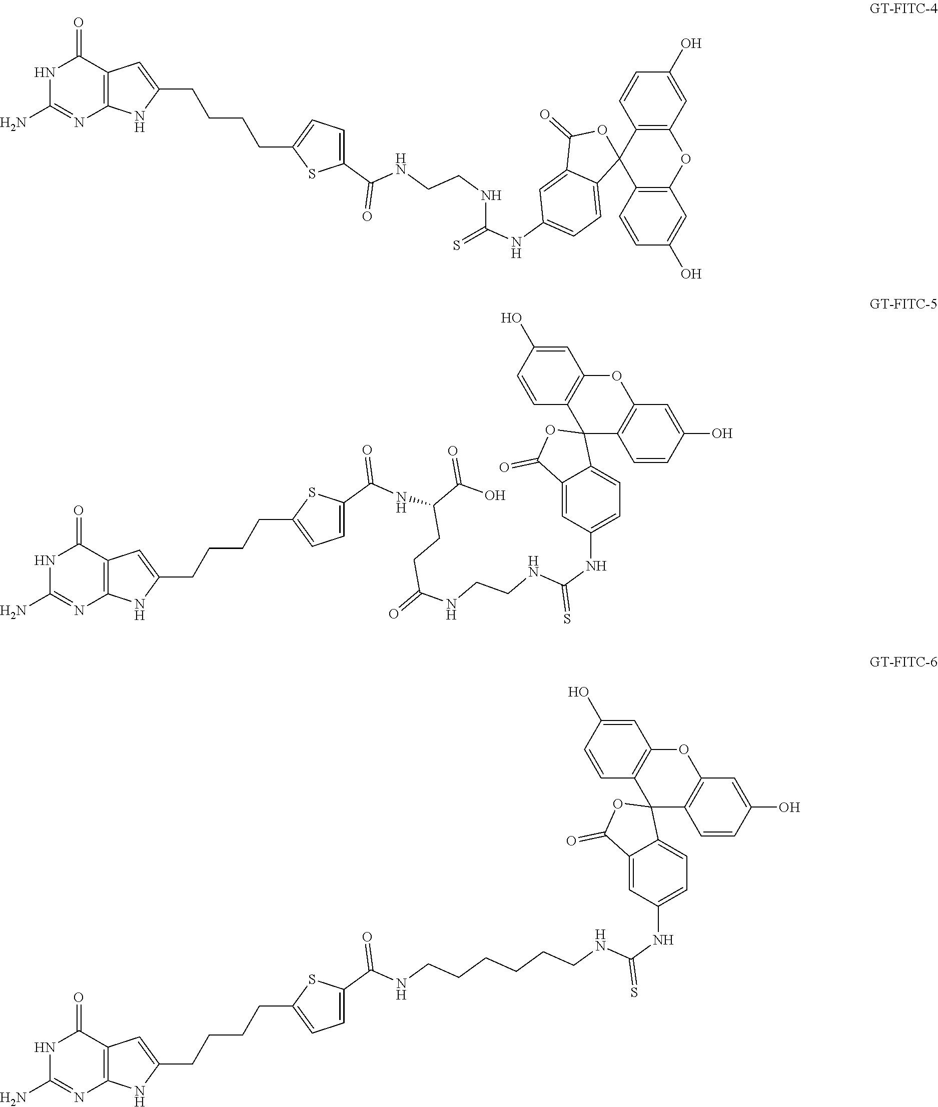 [SMILES: Nc1nc2[nH]c(CCCCc3ccc(C(=O)NCCNC(=S)Nc4ccc5c(c4)C(=O)OC54c5ccc(O)cc5Oc5cc(O)ccc54)s3)cc2c(=O)[nH]1 ; Nc1nc2[nH]c(CCCCc3ccc(C(=O)N[C@@H](CCC(=O)NCCNC(=S)Nc4ccc5c(c4)C(=O)OC54c5ccc(O)cc5Oc5cc(O)ccc54)C(=O)O)s3)cc2c(=O)[nH]1 ; Nc1nc2[nH]c(CCCCc3ccc(C(=O)NCCCCCCNC(=S)Nc4ccc5c(c4)C(=O)OC54c5ccc(O)cc5Oc5cc(O)ccc54)s3)cc2c(=O)[nH]1]

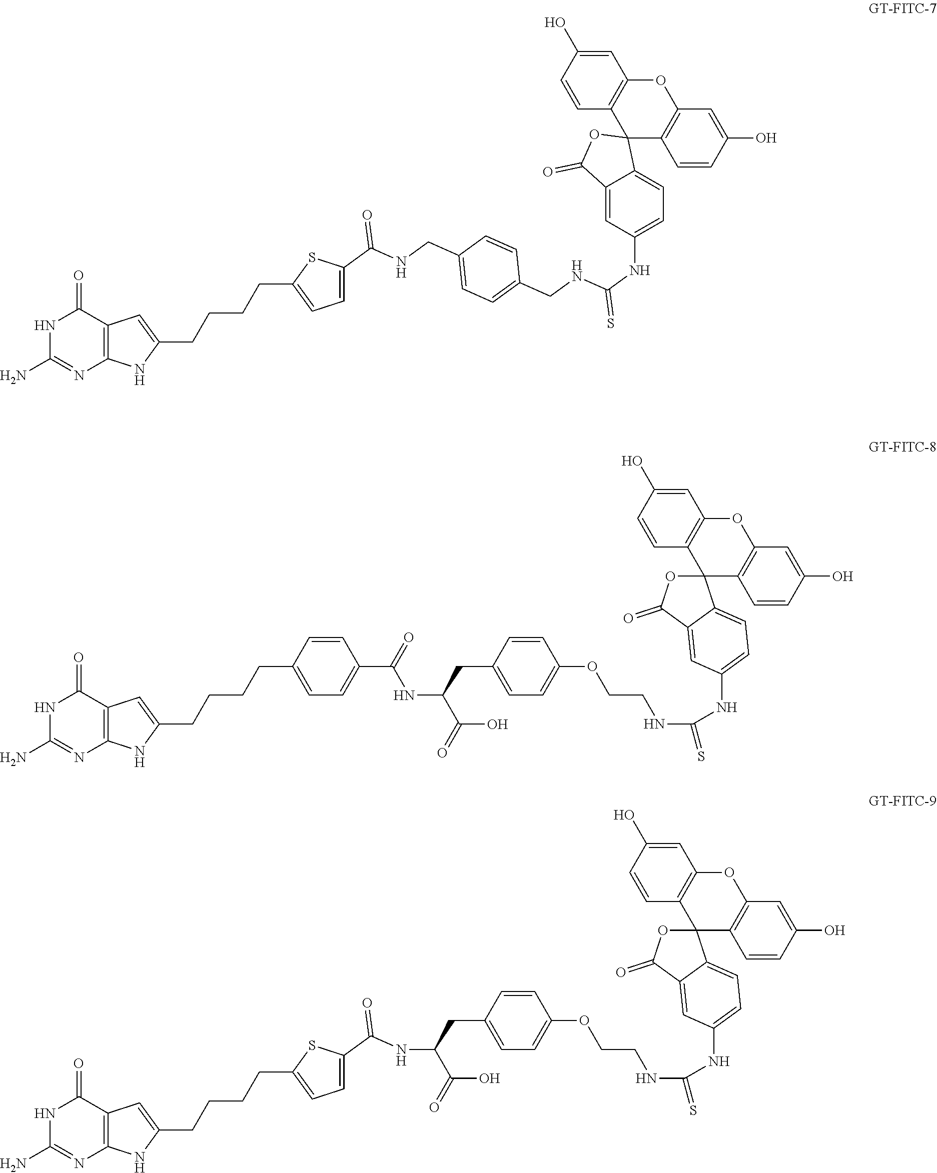 [SMILES: Nc1nc2[nH]c(CCCCc3ccc(C(=O)NCc4ccc(CNC(=S)Nc5ccc6c(c5)C(=O)OC65c6ccc(O)cc6Oc6cc(O)ccc65)cc4)s3)cc2c(=O)[nH]1 ; Nc1nc2[nH]c(CCCCc3ccc(C(=O)N[C@@H](Cc4ccc(OCCNC(=S)Nc5ccc6c(c5)C(=O)OC65c6ccc(O)cc6Oc6cc(O)ccc65)cc4)C(=O)O)cc3)cc2c(=O)[nH]1 ; Nc1nc2[nH]c(CCCCc3ccc(C(=O)N[C@@H](Cc4ccc(OCCNC(=S)Nc5ccc6c(c5)C(=O)OC65c6ccc(O)cc6Oc6cc(O)ccc65)cc4)C(=O)O)s3)cc2c(=O)[nH]1]

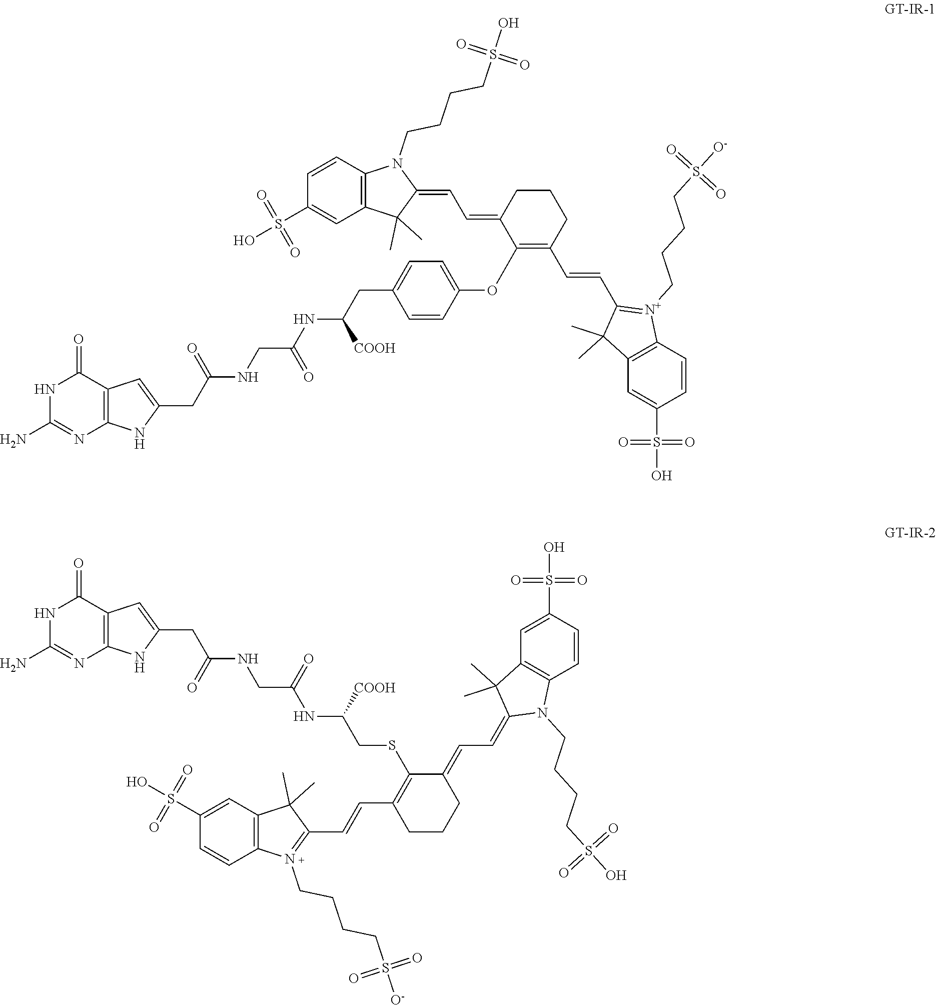

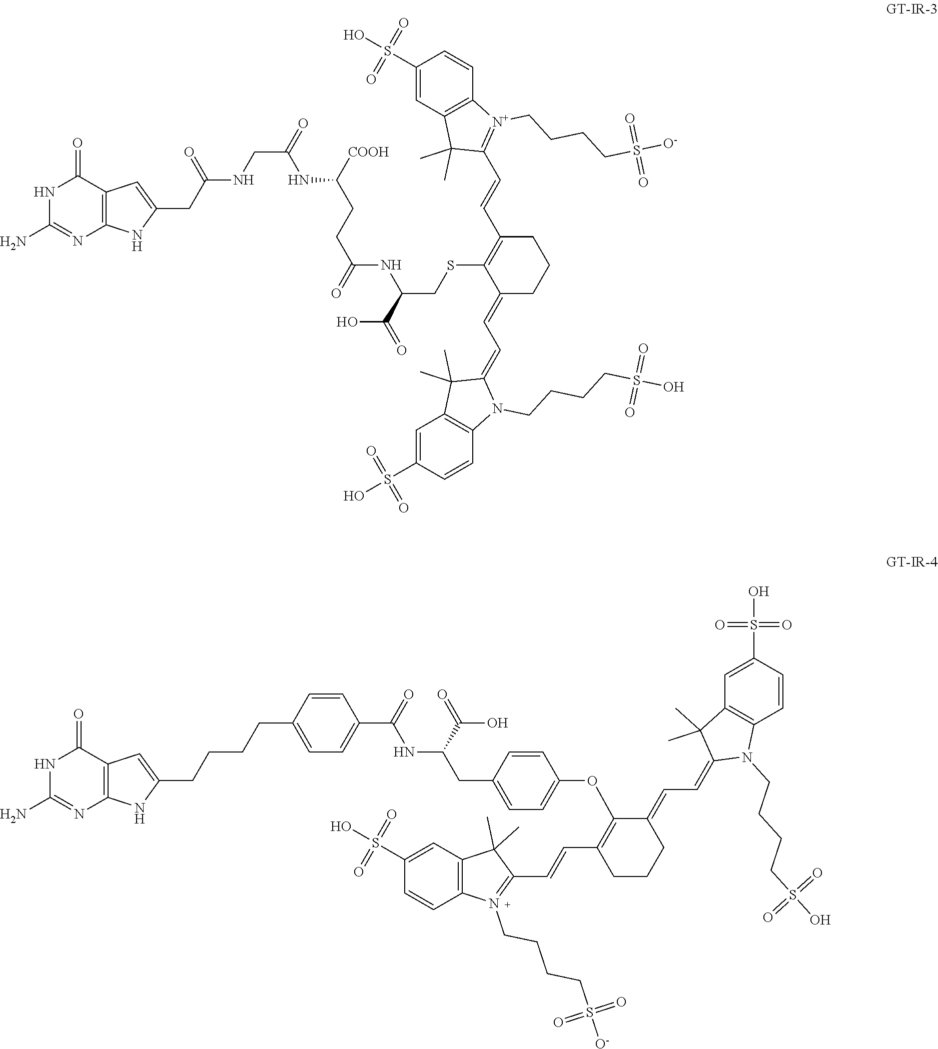

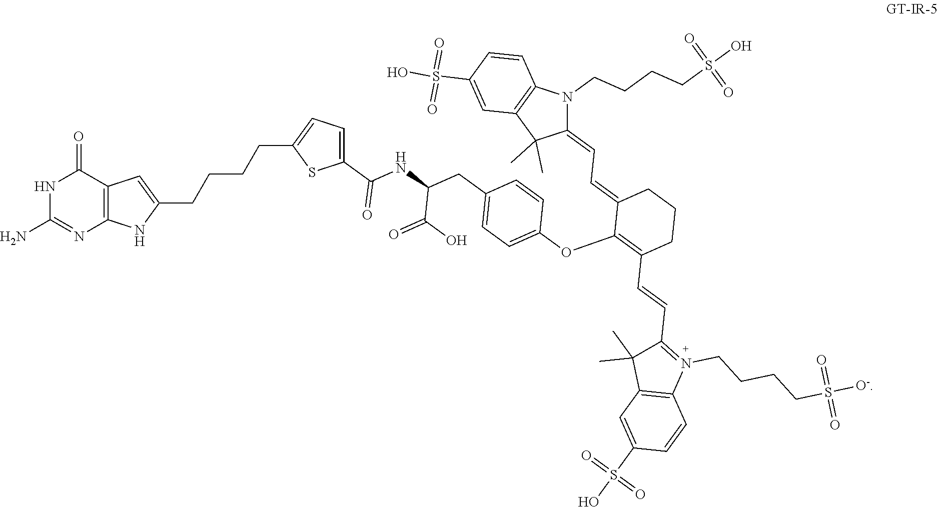

The term “pharmaceutically acceptable salt” as used herein refers to salts of the compounds of the present invention, which are prepared from relatively non-toxic acids or bases. When a compound of the present invention contains a relatively acidic functional group, a base addition salt may be obtained by contacting the neutral form of the compound with a sufficient amount of a base in a pure solution or a suitable inert solvent. Pharmaceutically acceptable base addition salts include, but are not limited to, those derived from inorganic bases and organic bases. Salts derived from inorganic bases include aluminum, ammonium, calcium, copper, ferric, ferrous, lithium, magnesium, manganic, manganous, potassium, sodium, zine salts, and the like. Salts derived from pharmaceutically acceptable organic non-toxic bases include salts of primary, secondary, and tertiary amines, as well as cyclic amines, such as benzylamine, dimethylamine, dicyclohexylamine, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucosamine, glucamine, histidine, hydrabamine, isopropylamine, lysine, methylglucamine, morpholine, piperazine, piperidine, polyamine resins, procaine, purines, theobromine, triethylamine, trimethylamine, tripropylamine, and the like. When a compound of the present invention contains a relatively basic functional group, an acid addition salt may be obtained by contacting the neutral form of the compound with a sufficient amount of an acid in a pure solution or a suitable inert solvent. Examples of pharmaceutically acceptable acid addition salts include those derived from inorganic acids such as hydrochloric acid, hydrobromic acid, nitric acid, carbonic acid, bicarbonate, phosphoric acid, monohydrogen phosphate, dihydrogen phosphate, sulfuric acid, hydrogen sulfate, hydroiodic acid, phosphorous acid, and the like; as well as salts derived from organic acids such as acetic acid, propionic acid, isobutyric acid, maleic acid, malonic acid, benzoic acid, succinic acid, suberic acid, fumaric acid, lactic acid, mandelic acid, phthalic acid, benzenesulfonic acid, p-toluenesulfonic acid, citric acid, tartaric acid, methanesulfonic acid, and similar acids.

In a second aspect, the present invention provides a composition comprising the aforementioned compound and at least one pharmaceutically acceptable carrier or excipient.

In a third aspect, the present invention provides a use of the aforementioned compound in the preparation of a tumor diagnostic agent for tumor-targeted imaging.

In some preferred embodiments, the tumor is selected from the group consisting of liver cancer, breast cancer, lung cancer, pancreatic cancer, colorectal cancer, and combinations thereof.

In a fourth aspect, the present invention provides a use of the compound in the preparation of an in vivo fluorescent imaging agent for precise tumor surgical navigation.

The technical solutions described above yield the following beneficial effects:

The fluorescent probe compound for tumor-targeted imaging provided by the present invention exhibits high affinity and selectivity for tumor cells expressing the target receptor. It enables the localization, qualitative, and quantitative analysis of target receptor-expressing tumor cells through in vitro and in vivo tracing, receptor affinity studies, and investigations into the mechanism of action. The compound demonstrates high specificity, sensitivity, and provides direct visualization. The fluorescent probe of the invention is rapidly cleared from normal tissues but remains retained at tumor sites for an extended period, thereby achieving in vivo diagnostic utility. It possesses considerable potential for clinical application, particularly in clinical intraoperative navigation.

### BRIEF DESCRIPTION OF DRAWINGS

FIG. 1 shows the absorption spectra of the GT-NIR series compounds prepared in the examples.

FIG. 2 shows the excitation spectra of the GT-NIR series compounds prepared in the examples.

FIG. 3 shows the emission spectra of the GT-NIR series compounds prepared in the examples.

FIG. 4 shows intracellular fluorescence imaging and localization of compound GT-NIR-1 in KB cells: (a) Upper and lower images show fluorescence imaging of GT-NIR-1 in KB cells and the corresponding blank image of KB cells, respectively; (b) Upper and lower images show fluorescence imaging of GT-NIR-1 co-incubated with 100-fold excess folic acid in KB cells and the corresponding blank image, respectively; (c) Upper and lower images show fluorescence imaging of GT-NIR-1 in A549 cells and the corresponding blank image of A549 cells, respectively.

FIG. 5 shows intracellular fluorescence imaging and localization of compound GT-NIR-1 in M2-type macrophages: (a) Upper and lower images show fluorescence imaging of GT-NIR-1 in M2-type macrophages and the corresponding blank image, respectively; (b) Upper and lower images show fluorescence imaging of GT-NIR-1 co-incubated with 100-fold excess folic acid in M2-type macrophages and the corresponding blank image, respectively; (c) Upper and lower images show fluorescence imaging of GT-NIR-1 in M1-type macrophages and the corresponding blank image, respectively.

FIG. 6 shows the binding affinity of the GT-NIR series compounds with KB cells measured by flow cytometry.

FIG. 7 shows the binding affinity of the GT-NIR series compounds with M2-type macrophages measured by flow cytometry.

FIG. 8 shows the equilibrium dissociation constant (K_{d}) of the GT-NIR series compounds with KB cells measured by flow cytometry.

FIG. 9 shows the equilibrium dissociation constant (K_{d}) of the GT-NIR series compounds with M2-type macrophages measured by flow cytometry.

FIG. 10 shows the absorption spectra of the GT-FITC series compounds: (a) Absorption spectra of FITC and GT-FITC-1˜4; (b) Absorption spectra of GT-FITC-5˜9.

FIG. 11 shows the excitation spectra of the GT-FITC series compounds: (a) Excitation spectra of FITC and GT-FITC-1˜4; (b) Excitation spectra of GT-FITC-5˜9.

FIG. 12 shows the emission spectra of the GT-FITC series compounds: (a) Emission spectra of FITC and GT-FITC-1˜4; (b) Emission spectra of GT-FITC-5˜9.

FIG. 13 shows the binding affinity of the GT-FITC series compounds with KB cells measured by flow cytometry: (a) Flow cytometry results of FITC and GT-FITC-1˜4; (b) Flow cytometry results of GT-FITC-5˜9.

FIG. 14 shows quantitative fluorescence analysis of the GT-NIR series compounds (a) and GT-IR series compounds (b) in intracellular fluorescence imaging localization experiments; wherein KB represents folate receptor-positive KB cells, A549 represents folate receptor-negative A549 cells, and Blocked represents co-incubation of KB cells with 100-fold excess folic acid and the probe.

FIG. 15 shows whole-body fluorescence imaging after injection of the GT-NIR series compounds into KB cell xenograft mouse models.

FIG. 16 shows whole-body fluorescence imaging after injection of compound GT-NIR-4 into A549 cell xenograft mouse models (A549), and co-injection of 100× excess folic acid (FA) with GT-NIR-4 into KB cell xenograft mouse models (100×FA).

FIG. 17 shows ex vivo tissue distribution of the GT-NIR series compounds after injection into KB cell xenograft mouse models. Tissues from top to bottom are: tumor, heart, lung, liver, spleen, stomach, kidney, and intestine: (a) GT-NIR-1; (b) GT-NIR-2; (c) GT-NIR-3; (d) GT-NIR-4; (e) GT-NIR-5; (f) 100×FA+GT-NIR-4.

FIG. 18 shows ex vivo tissue distribution of compound GT-NIR-4 after injection into A549 cell xenograft mouse models, wherein tissues from top to bottom are: tumor, heart, lung, liver, spleen, stomach, kidney, and intestine.

FIG. 19 shows whole-body fluorescence imaging and corresponding ex vivo tissue distribution of the GT-IR series compounds after injection into KB cell xenograft mouse models.

FIG. 20 shows the effect of the GT-NIR series compounds on HUVEC cell viability.

### DETAILED DESCRIPTION OF THE EMBODIMENTS

To make the objectives, technical solutions, and advantages of the present invention clearer, the invention is described below clearly and completely in conjunction with specific embodiments.

### Example 1: Synthesis of Substrate I-1

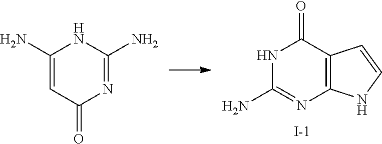 [SMILES: Nc1cc(=O)nc(N)[nH]1 ; Nc1nc2[nH]ccc2c(=O)[nH]1 ; [I-]]

2,6-Diaminopyrimidin-4(1H)-one (50 g, 0.4 mol) and sodium acetate (33.6 g, 0.4 mol) were added to water (150 mL). The mixture was heated to reflux, followed by the dropwise addition of chloroacetaldehyde (37.6 g, 0.48 mol). After refluxing for 18 hours, the reaction mixture was cooled to room temperature. The resulting precipitate was collected by filtration, washed successively with water (2×50 mL) and acetone (2×50 mL), and dried to afford Substrate I-1. ESI-MS(m/z): 151.1 [M+H]⁺.

### Example 2: Synthesis of Substrate I-2

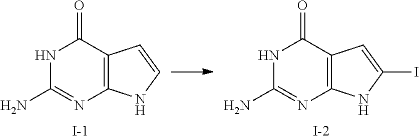

Substrate I-1 (1.5 g, 0.01 mol) and iodine (5.1 g, 0.02 mol) were dissolved in a mixture of ethanol/water (2:1, 100 mL). The solution was heated to reflux for 2 hours. After cooling, the precipitated crystals were collected by filtration, washed successively with 1N sodium thiosulfate solution (2×30 mL) and water (2×50 mL), and dried to afford Substrate I-2. ESI-MS(m/z) 276.9 [M+H]⁺.

### Example 3: Synthesis of Substrate I-3

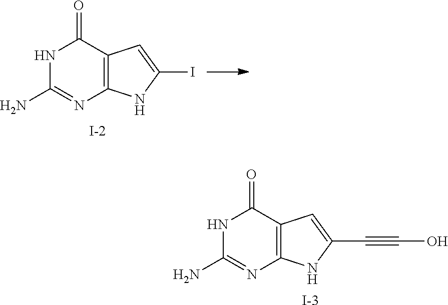

Substrate I-2 (2.7 g, 0.01 mol) and ethynyl alcohol (0.43 g, 0.01 mol) were dissolved in anhydrous DMF (30 mL). To the solution were added palladium(II) chloride (71 mg, 0.40 mmol), triphenylphosphine (131 mg, 0.40 mmol), triethylamine (10.1 g, 0.1 mol), and copper(I) iodide (304 mg, 1.60 mmol). The reaction mixture was heated at 100° C. for 12 hours. After completion, the solvent was removed under reduced pressure, and the crude product was purified by column chromatography to afford Substrate I-3. ESI-MS(m/z): 191.1 [M+H]⁺.

### Example 4: Synthesis of Substrate I-4

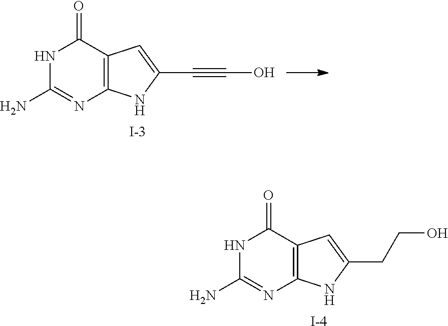

Substrate I-3 (1.9 g, 0.01 mol) was dissolved in methanol (50 mL), followed by the addition of 5% palladium on carbon (200 mg). The reaction was subjected to catalytic hydrogenation at 50 psi under room temperature for 12 hours. The mixture was filtered to remove the catalyst, and the filtrate was concentrated under reduced pressure to yield Substrate I-4. ESI-MS(m/z): 195.1 [M+H]⁺.

### Example 5: Synthesis of Substrate I-5

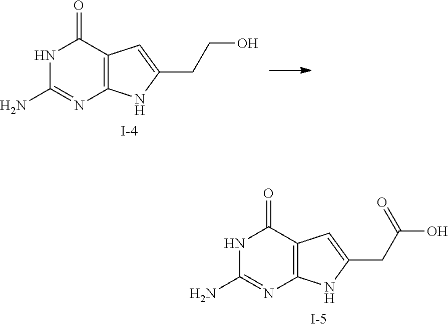

Substrate I-4 (2 g, 0.01 mol) was dissolved in acetone (20 mL) and cooled in an ice bath. A mixture of chromium trioxide (6 g, 0.06 mol), sulfuric acid (30 mL), and water (90 mL) was added dropwise. After complete addition, the reaction was continued in the ice bath for 2 hours and then allowed to warm to room temperature overnight. The mixture was extracted with ethyl acetate (5×30 mL), and the combined organic layers were dried over anhydrous sodium sulfate. The solvent was evaporated under reduced pressure, and the residue was purified by column chromatography to give Substrate I-5. ESI-MS(m/z): 207.1 [M+H]⁺.

### Example 6: Synthesis of Substrate I-6

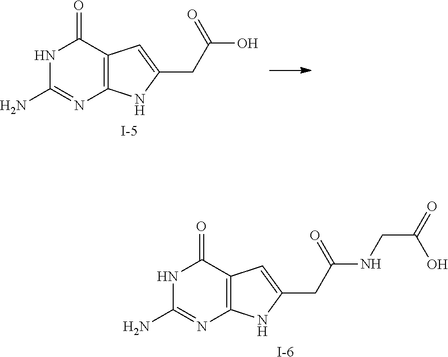

Substrate I-5 (1.0 g, 5 mmol) and 1-hydroxybenzotriazole (0.8 g, 6 mmol) were dissolved in anhydrous DMF (20 mL) and stirred at room temperature for 2 hours. Subsequently, 1-ethyl-(3-dimethylaminopropyl)carbodiimide hydrochloride (1.2 g, 6 mmol), glycine methyl ester hydrochloride (1.3 g, 10 mmol), and triethylamine (1.0 g, 10 mmol) were added. The reaction was continued at room temperature for 5 hours. The solvent was then removed under reduced pressure, and Na₂CO₃ solution (50 mL) was added to precipitate the product. The precipitate was collected by filtration, dissolved in 1N NaOH (100 mL), and stirred at room temperature for 1.5 hours. The solution was cooled in an ice bath, and the pH was adjusted to 2-3 using 1N HCl. The resulting precipitate was filtered, washed with cold water (30 mL), and dried to afford Substrate I-6 (860 mg, 65% yield). ¹H NMR (600 MHz, DMSO-d₆): 12.24 (br, 1H), 10.81 (s, 1H), 10.18 (s, 1H), 8.10-8.13 (t, J=5.6 Hz, 1H), 6.03 (s, 2H), 6.02 (s, 1H), 3.75-3.77 (d, J=5.6 Hz, 2H), 6=3.41 (s, 2H); ESI-MS(m/z): 264.1 [M−H]⁻.

### Example 7: Synthesis of Substrate I-7

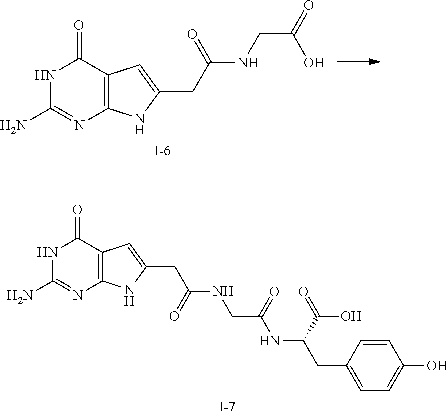

Substrate I-6 (670 mg, 1.0 eq), 2-(7-azabenzotriazol-1-yl)-N,N,N′,N′-tetramethyluronium hexafluorophosphate (1.15 g, 1.2 eq), O-tert-butyl-L-tyrosine tert-butyl ester hydrochloride (1.0 g, 1.2 eq), and N,N-diisopropylethylamine (1.3 g, 4 eq) were sequentially dissolved in anhydrous DMF (30 mL) and reacted at room temperature for 4 hours. The solvent was removed under reduced pressure, and the crude product was purified by column chromatography. The purified intermediate (480 mg, 1.0 eq) was dissolved in anhydrous dichloromethane, followed by the sequential addition of trifluoroacetic acid (3 mL, 50 eq) and triethylsilane (0.8 mL, 5.0 eq). The mixture was stirred at room temperature for 3 hours. After removal of the solvent under reduced pressure, Substrate I-7 was obtained (240 mg, 63.1% yield). ¹H NMR (600 MHz, DMSO-d₆): δ 11.19 (s, 1H), 8.13 (d, J=8.0 Hz, 1H), 8.06 (t, J=5.6 Hz, 2H), 6.99 (d, J=8.3 Hz, 2H), 6.64 (d, J=8.3 Hz, 2H), 6.08 (s, 1H), 4.35 (td, J=8.4, 5.4 Hz, 1H), 3.70 (ddd, J=52.2, 16.8, 5.7 Hz, 2H), 3.44 (s, 2H), 2.96-2.67 (m, 2H); ¹³C NMR (151 MHz, DMSO-d₆) δ 174.40, 169.85, 168.24, 159.24, 155.96, 152.88, 151.74, 130.69, 129.26, 125.14, 115.17, 100.78, 100.29, 55.85, 42.91, 37.24, 35.32; HRMS(APCI): Calcd for C₁₉H₂₀N₆O₆ [M+H]⁺: 429.1517, found 429.1498.

### Example 8: Synthesis of Substrate II-1

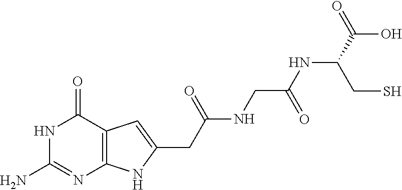 [SMILES: Nc1nc2[nH]c(CC(=O)NCC(=O)N[C@@H](CS)C(=O)O)cc2c(=O)[nH]1]

Following a procedure analogous to that described in Example 7, Substrate II-1 was obtained as a light-yellow solid (180 mg, 58.9% yield). ¹H NMR (600 MHz, DMSO-d₆) δ 10.84 (s, 1H), 8.25 (t, J=5.9 Hz, 1H), 7.76 (d, J=6.6 Hz, 1H), 6.21 (s, 2H), 6.02 (s, 1H), 4.07 (q, J=5.4 Hz, 1H), 3.73 (dd, J=5.9, 1.9 Hz, 2H), 3.45 (s, 2H), 2.83 (ddd, J=48.5, 13.1, 5.0 Hz, 2H); ¹³C NMR (151 MHz, DMSO-d₆) δ 172.54, 172.06, 168.87, 168.73, 159.11, 152.72, 151.72, 125.18, 125.10, 100.82, 100.35, 55.69, 42.97, 40.40, 40.27, 40.13, 39.99, 39.85, 39.71, 39.57, 35.32, 27.10; HRMS(APCI): Calcd for C₁₃H₁₆N₆O₅S [M+H]⁺: 369.0976, found 369.0968.

### Example 9: Synthesis of Substrate III-1

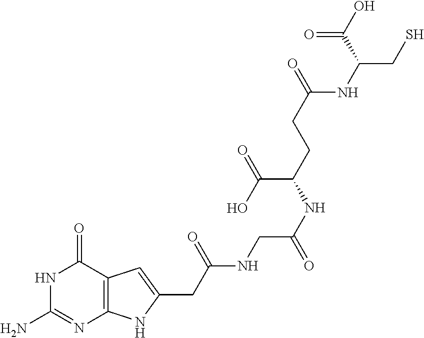 [SMILES: Nc1nc2[nH]c(CC(=O)NCC(=O)N[C@@H](CCC(=O)N[C@@H](CS)C(=O)O)C(=O)O)cc2c(=O)[nH]1]

Following a procedure analogous to that described in Example 7, Substrate III-1 was obtained as a light-yellow solid (120 mg, 41.5%). ¹H NMR (600 MHz, DMSO-d₆) δ 10.80 (s, 1H), 10.19 (s, 1H), 8.34 (d, J=7.8 Hz, 1H), 8.24 (d, J=7.6 Hz, 1H), 8.03 (t, J=5.7 Hz, 1H), 6.05 (s, 2H), 6.00 (s, 1H), 4.13 (q, J=7.3 Hz, 1H), 4.05-3.92 (m, 1H), 3.84-3.62 (m, 2H), 3.41 (s, 2H), 2.46-2.27 (m, 2H), 2.19 (t, J=8.0 Hz, 2H), 1.97-1.71 (m, 2H); HRMS(APCI): Calcd for C₁₈H₂₃N₇O₈S [M+H]⁺: 498.1402, found 498.1390.

### Example 10: Synthesis of Substrate IV-1

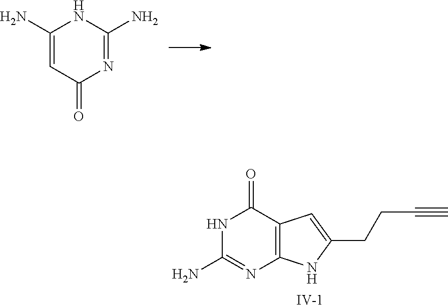

To a suspension of 2,6-diaminopyrimidin-4-one (1.26 g, 10 mmol) in anhydrous DMF (25 mL) was added 1-bromo-5-hexyn-2-one (0.98 g, 10 mmol). The resulting mixture was stirred under N₂ at room temperature for 3 days. After evaporation of the solvent under reduced pressure, the crude product was purified by column chromatography to afford Substrate IV-1 (1.4 g, 74.0% yield). ¹H NMR (600 MHz, DMSO-d₆) 10.81 (s, 1H), 10.13 (s, 1H), 5.98 (s, 2H), 5.93 (s, 1H), 2.77 (t, J=2 Hz, 1H), 2.64-2.67 (m, 2H), 62.41-2.45 (m, 2H); HRMS(APCI): Calcd for C₁₀H₁₀N₄O [M+H]⁺: 203.0927, found, 203.0935.

### Example 11: Synthesis of Substrate IV-2

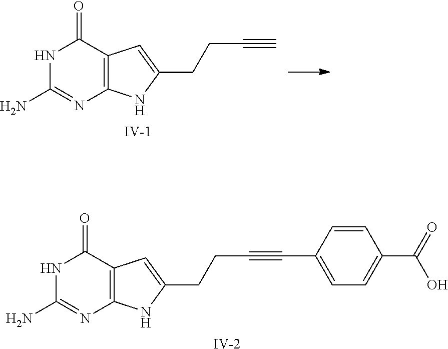

In a 250 mL round-bottom flask equipped with a magnetic stirrer and gas inlet, tetrakis(triphenylphosphine)palladium(0) (185 mg, 0.16 mmol), triethylamine (1.01 g, 10 mmol), methyl 4-iodobenzoate (393 mg, 1.5 mmol), and anhydrous DMF (20 mL) were combined. To the stirred mixture under N₂, copper(I) iodide (30 mg, 0.16 mmol) and Substrate IV-1 (202 mg, 1 mmol) were added, and the reaction mixture was stirred at room temperature overnight. After evaporation of the solvent under reduced pressure, the residue was purified by column chromatography. The resulting solid was dissolved in 1N NaOH (100 mL) and stirred at room temperature for 1.5 hours. The solution was cooled in an ice bath, and the pH was adjusted to 2-3 with 1N HCl. The precipitated solid was collected by filtration, washed with cold water (30 mL), and dried to afford Substrate IV-2 (238 mg, 74.0% yield). ¹H NMR (600 MHz, DMSO-d₆) δ 10.79 (s, 1H), 10.21 (s, 1H), 7.86 (d, J=8.4 Hz, 2H), 7.32 (d, J=8.4 Hz, 2H), 6.02 (s, 2H), 5.85 (s, 1H), 2.85 (t, J=7.0 Hz, 2H), 1.62 (t, J=6.4 Hz, 2H); HRMS(APCI): Calcd for C₁₇H₁₄N₄O₃ [M+H]⁺: 323.1139, found, 323.1152.

### Example 12: Synthesis of Substrate IV-3

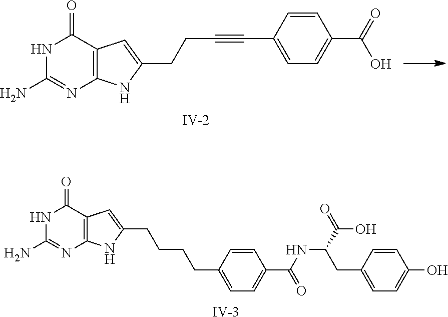 [SMILES: Nc1nc2[nH]c(CCC#Cc3ccc(C(=O)O)cc3)cc2c(=O)[nH]1 ; "IV-2" ; Nc1nc2[nH]c(CCCCc3ccc(C(=O)N[C@@H](Cc4ccc(O)cc4)C(=O)O)cc3)cc2c(=O)[nH]1 ; "IV-3"]

Substrate IV-2 (322 mg, 1 mmol) was dissolved in methanol (30 mL), and 5% palladium on carbon (100 mg) was added. The mixture was subjected to catalytic hydrogenation at 50 psi and room temperature for 12 hours. After filtration and removal of the solvent under reduced pressure, a white solid was obtained. Using a procedure analogous to that described in Example 7, Substrate IV-3 was isolated (190 mg, 80.6% yield). ¹H NMR (600 MHz, DMSO-d₆) δ 10.78 (s, 1H), 10.23 (s, 1H), 9.18 (s, 1H), 8.38 (d, J=8.0 Hz, 1H), 7.69 (d, J=8.0 Hz, 2H), 7.25 (d, J=8.0 Hz, 2H), 7.06 (d, J=8.3 Hz, 2H), 6.67-6.59 (m, 2H), 6.01 (s, 2H), 5.85 (s, 1H), 4.46 (ddd, J=10.0, 7.9, 4.5 Hz, 1H), 3.08-2.90 (m, 2H), 2.62 (d, J=7.1 Hz, 2H), 1.62-1.51 (m, J=3.7 Hz, 4H); ¹³C NMR (151 MHz, DMSO-d₆) δ 173.98, 166.51, 158.96, 156.18, 152.36, 151.56, 146.25, 132.21, 131.49, 130.48, 128.90, 128.62, 127.78, 115.37, 100.12, 98.31, 55.29, 36.16, 35.16, 30.76, 28.81, 27.50; HRMS(APCI): Calcd for C₂₆H₂₇N₅O₅ [M+H]⁺: 490.2085, found, 490.2067.

### Example 13: Synthesis of Substrate IV-4

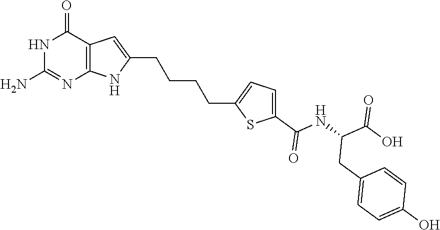 [SMILES: Nc1nc2[nH]c(CCCCc3ccc(C(=O)N[C@@H](Cc4ccc(O)cc4)C(=O)O)s3)cc2c(=O)[nH]1]

Following a procedure analogous to that described in Example 12, Substrate IV-4 was obtained (160 mg, 74.3% yield). ¹H NMR (600 MHz, DMSO-d₆) δ 10.79 (s, 1H), 7.94 (d, J=7.8 Hz, 1H), 7.51 (d, J=3.7 Hz, 1H), 6.99 (d, J=8.0 Hz, 2H), 6.81 (d, J=3.7 Hz, 1H), 6.58 (d, J=8.0 Hz, 2H), 6.24 (s, 2H), 5.86 (s, 1H), 4.26 (td, J=8.1, 4.5 Hz, 1H), 2.96 (ddd, J=113.8, 13.7, 6.4 Hz, 2H), 2.77 (d, J=7.0 Hz, 2H), 1.61 (p, J=3.7 Hz, 4H); ¹³C NMR (151 MHz, DMSO-d₆) δ 174.59, 160.80, 159.19, 155.96, 152.63, 151.71, 150.06, 137.96, 131.34, 130.54, 129.65, 128.28, 125.71, 115.20, 100.06, 98.33, 56.35, 49.07, 45.72, 36.96, 31.04, 29.72, 28.61, 27.38; HRMS(APCI): Calcd for C₂₄H₂₅N₅O₅S [M+H]⁺: 496.1649, found, 496.1633.

### Example 14: Synthesis of Compound GT-NIR-1

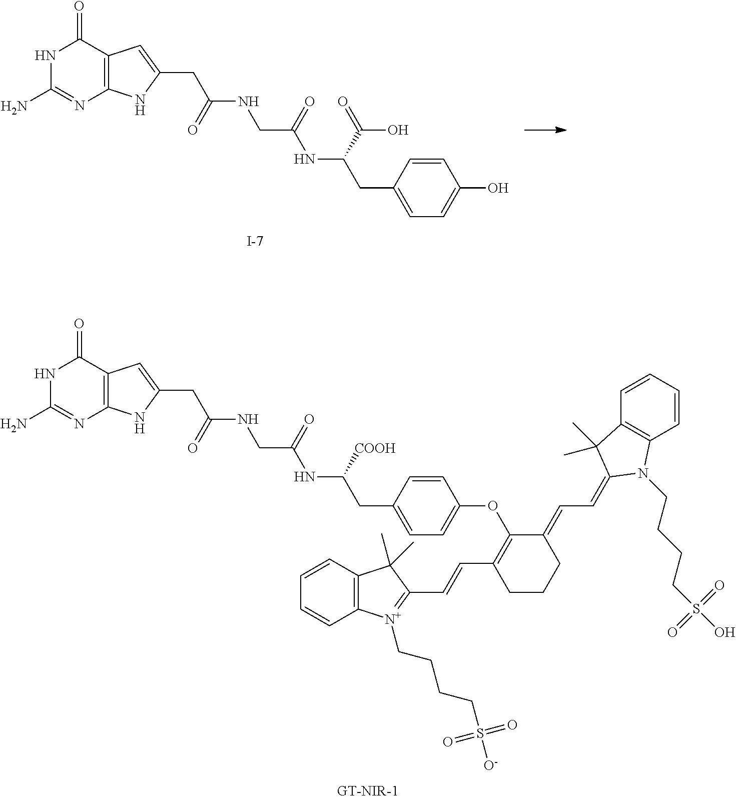

Substrate I-7 (42 mg, 0.1 mmol) was dissolved in 1N NaOH (10 mL) and stirred at room temperature for 30 minutes. IR-783 (75 mg, 0.1 mmol) was added, and the reaction was heated to 85° C. The reaction progress was monitored by HPLC. After completion, the crude product was purified by preparative liquid chromatography. The target fractions were lyophilized to afford GT-NIR-1 as a dark green solid (34 mg, 28.9% yield). ¹H NMR (600 MHz, DMSO-d₆) δ 10.85 (s, 1H), 8.19 (s, 1H), 7.81 (d, J=13.9 Hz, 2H), 7.43 (d, J=7.5 Hz, 2H), 7.35 (t, J=8.5 Hz, 3H), 7.32 (d, J=7.7 Hz, 2H), 7.16 (d, J=8.4 Hz, 2H), 7.13 (t, J=7.2 Hz, 2H), 6.97 (d, J=8.4 Hz, 2H), 6.27 (t, J=19.0 Hz, 2H), 6.19 (d, J=14.0 Hz, 2H), 5.97 (s, 1H), 4.11 (t, J=7.4 Hz, 4H), 4.05-4.00 (m, 1H), 3.57 (dd, J=17.8, 6.8 Hz, 2H), 3.37-3.27 (m, 2H), 3.03-2.80 (m, 2H), 2.70 (t, J=6.5 Hz, 4H), 1.78-1.66 (m, 10H), 1.24 (dd, J=15.6, 4.7 Hz, 12H); ¹³C NMR (151 MHz, DMSO-d₆) δ 174.82, 172.58, 169.79, 163.23, 159.05, 158.53, 152.65, 151.66, 142.52, 141.45, 131.50, 130.12, 128.87, 125.14, 122.81, 122.05, 114.43, 111.67, 100.67, 100.36, 51.19, 48.99, 43.98, 35.58, 35.24, 31.75, 29.55, 29.49, 29.44, 29.30, 29.21, 29.16, 29.04, 27.74, 27.69, 27.02, 26.47, 25.58, 24.15, 22.93, 22.56, 21.62, 14.42; HRMS(APCI): Calcd for C₅₇H₆₆N₈O₁₂S₂[M−H]⁻: 1117.4169, found, 1117.4174.

### Example 15: Synthesis of Compound GT-NIR-2

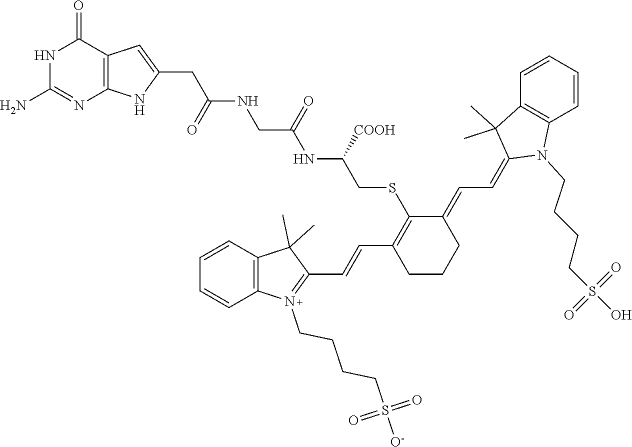

Following a procedure analogous to that described in Example 14, GT-NIR-2 was obtained as a dark green solid (22 mg, 34.3% yield). ¹H NMR (600 MHz, DMSO-d₆) δ 10.85 (s, 1H), 8.67 (d, J=13.9 Hz, 2H), 8.11 (d, J=5.7 Hz, 1H), 7.70 (d, J=6.2 Hz, 1H), 7.53 (d, J=7.4 Hz, 2H), 7.40 (d, J=8.0 Hz, 2H), 7.37 (t, J=7.6 Hz, 2H), 7.21 (t, J=7.2 Hz, 2H), 6.26 (d, J=14.2 Hz, 2H), 6.23 (s, 2H), 6.03 (s, 1H), 4.16 (t, J=7.7 Hz, 4H), 4.05 (q, J=5.6 Hz, 1H), 3.68 (qd, J=16.7, 5.6 Hz, 2H), 3.44 (d, J=3.0 Hz, 2H), 3.34-3.11 (m, 2H), 2.54 (t, J=7.4 Hz, 4H), 1.81 (q, J=7.4 Hz, 4H), 1.74 (hept, J=6.5, 6.1 Hz, 6H), 1.67 (s, 12H); ¹³C NMR (151 MHz, DMSO-d₆) δ 172.70, 171.99, 169.83, 168.58, 159.11, 158.69, 152.75, 151.66, 145.18, 142.76, 141.49, 133.09, 128.90, 125.17, 125.04, 122.83, 111.58, 101.52, 100.79, 100.38, 55.41, 51.23, 49.21, 43.99, 42.85, 41.28, 35.33, 29.49, 28.11, 27.97, 27.02, 26.47, 26.13, 22.99, 21.79, 21.00; HRMS(APCI): Calcd for C₅₁H₆₂N₈O₁₁S₃[M−H]⁻: 1057.3627, found, 1057.3590.

### Example 16: Synthesis of Compound GT-NIR-3

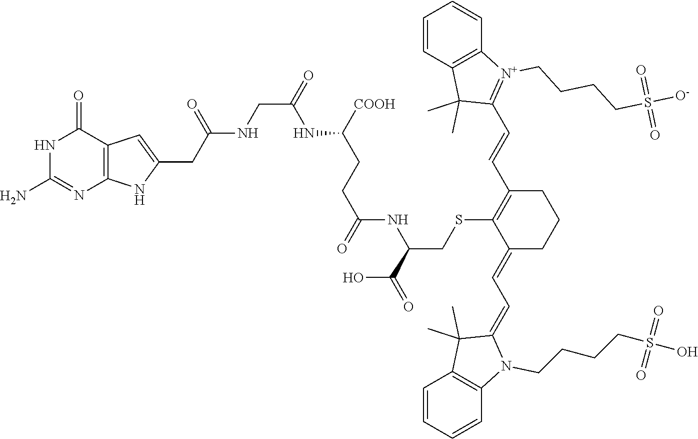 [SMILES: CC1(C)C(/C=C/C2=C(SC[C@H](NC(=O)CC[C@H](NC(=O)CNC(=O)Cc3cc4c(=O)[nH]c(N)nc4[nH]3)C(=O)O)C(=O)O)C(=C/C=C3/N(CCCCS(=O)(=O)O)c4ccccc4C3(C)C)/CCC2)=[N+](CCCCS(=O)(=O)[O-])c2ccccc21]

Following a procedure analogous to that described in Example 14, GT-NIR-3 was obtained as a dark green solid (22 mg, 31.2%). ¹H NMR (600 MHz, DMSO-d₆) δ 11.00 (s, 1H), 8.67 (d, J=13.7 Hz, 2H), 8.36 (t, J=6.0 Hz, 1H), 7.65 (d, J=6.9 Hz, 1H), 7.58 (d, J=7.3 Hz, 2H), 7.53 (d, J=6.3 Hz, 1H), 7.39 (d, J=8.1 Hz, 2H), 7.37 (t, J=7.5 Hz, 2H), 7.20 (t, J=7.2 Hz, 2H), 6.40 (s, 2H), 6.27 (d, J=13.9 Hz, 2H), 6.02 (s, 1H), 4.17 (d, J=8.8 Hz, 4H), 4.11 (d, J=6.8 Hz, 1H), 3.91 (q, J=6.4 Hz, 1H), 3.71-3.58 (m, 2H), 3.45 (s, 2H), 3.33-3.12 (m, 2H), 2.56 (t, J=7.4 Hz, 5H), 2.16-2.03 (m, 2H), 1.95-1.89 (m, 2H), 1.80 (q, J=7.4 Hz, 4H), 1.75 (q, J=7.8, 7.2 Hz, 6H), 1.67 (s, 12H); ¹³C NMR (151 MHz, DMSO-d₆) δ 174.61, 172.86, 172.64, 172.06, 171.95, 170.07, 168.51, 159.28, 152.91, 151.72, 145.19, 142.73, 141.48, 133.12, 128.89, 125.26, 125.05, 122.92, 111.53, 101.52, 100.86, 100.29, 55.47, 53.96, 51.22, 49.20, 43.96, 43.09, 41.21, 35.35, 32.86, 28.81, 28.07, 27.99, 26.46, 26.17, 22.96, 22.01, 20.98; HRMS(APCI): Calcd for C₅₆H₆₉N₉O₁₄S₃[M−H]⁻: 1186.4053, found, 1186.4015.

### Example 17: Synthesis of Compound GT-NIR-4

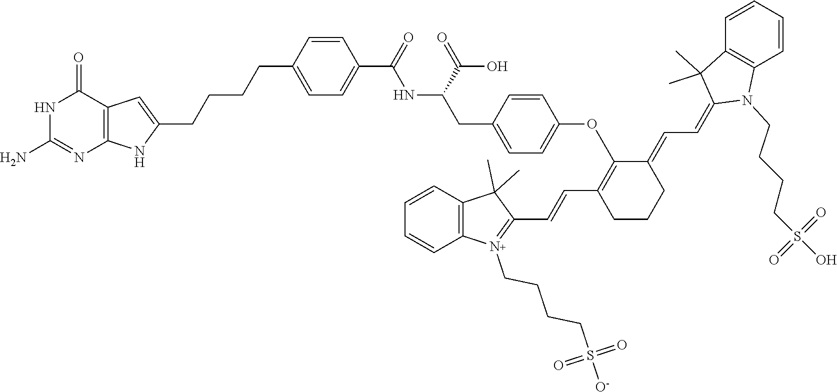

Following a procedure analogous to that described in Example 14, GT-NIR-4 was obtained as a dark green solid (20 mg, 24.3%). ¹H NMR (600 MHz, DMSO-d₆) δ 10.82 (s, 1H), 7.91 (s, 1H), 7.76 (d, J=13.9 Hz, 2H), 7.57 (d, J=7.4 Hz, 2H), 7.33 (s, 2H), 7.28 (d, J=8.0 Hz, 4H), 7.20 (t, J=7.3 Hz, 2H), 7.12 (t, J=6.8 Hz, 2H), 7.08 (d, J=7.8 Hz, 2H), 6.98 (d, J=7.8 Hz, 2H), 6.24 (s, 2H), 6.15 (d, J=14.1 Hz, 2H), 5.83 (s, 1H), 4.29 (s, 1H), 4.08 (s, 4H), 3.20-2.93 (m, 2H), 2.67 (s, 4H), 2.57-2.52 (m, 5H), 1.74 (d, J=23.7 Hz, 8H), 1.61-1.51 (m, 2H), 1.48 (q, J=7.7 Hz, 2H), 1.26-1.20 (m, 2H), 1.12 (s, 12H); ¹³C NMR (151 MHz, DMSO-d₆) δ 172.09, 171.86, 165.87, 163.30, 158.33, 152.58, 151.70, 145.89, 142.42, 141.44, 141.28, 133.58, 132.74, 131.45, 128.88, 128.39, 127.51, 125.05, 122.61, 122.06, 114.21, 111.60, 100.57, 100.05, 98.29, 51.18, 48.83, 43.90, 40.39, 40.12, 39.98, 39.84, 39.70, 39.56, 36.61, 35.10, 30.60, 29.49, 28.78, 27.69, 27.59, 27.44, 26.43, 25.94, 24.10, 22.96, 22.91, 22.87, 22.56, 22.20, 21.19; HRMS(APCI): Calcd for C₆₄H₇₃N₇O₁₁S₂[M−H]⁻: 1178.4737, found, 1178.4705.

### Example 18: Synthesis of Compound GT-NIR-5

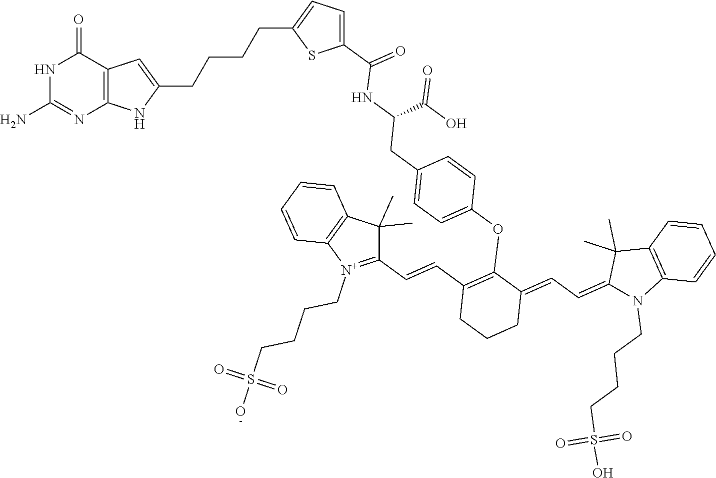

Following a procedure analogous to that described in Example 14, GT-NIR-3 was obtained as a dark green solid (22 mg, 23.3%). ¹H NMR (600 MHz, DMSO-d₆) δ 10.82 (s, 1H), 8.29-8.25 (m, 1H), 7.76 (d, J=13.9 Hz, 2H), 7.49 (d, J=3.9 Hz, 1H), 7.42-7.20 (m, 8H), 7.15 (t, J=7.0 Hz, 2H), 7.02 (d, J=8.0 Hz, 2H), 6.71 (d, J=3.9 Hz, 1H), 6.17 (d, J=14.1 Hz, 2H), 6.09 (s, 2H), 5.85 (s, 1H), 4.37 (t, J=10.3 Hz, 1H), 4.09 (s, 4H), 3.15-2.89 (m, 2H), 2.69 (s, 4H), 2.65-2.57 (m, 2H), 2.48 (s, 4H), 1.71 (d, J=17.2 Hz, 8H), 1.57 (p, J=7.1 Hz, 2H), 1.47 (q, J=7.7 Hz, 2H), 1.23 (s, 4H), 1.14 (s, 12H); ¹³C NMR (151 MHz, DMSO-d₆) δ 172.58, 171.89, 163.21, 161.37, 159.01, 158.49, 152.43, 151.63, 150.46, 142.43, 141.41, 141.31, 137.33, 132.93, 131.38, 131.31, 130.12, 128.88, 128.64, 125.44, 125.05, 122.66, 122.03, 114.41, 111.62, 100.61, 100.12, 98.35, 55.54, 51.19, 49.07, 48.86, 43.93, 36.18, 35.59, 31.75, 30.73, 29.64, 29.49, 29.29, 29.16, 28.53, 27.61, 27.53, 27.26, 27.02, 26.46, 24.87, 24.11, 22.92, 22.56, 21.64, 21.20, 14.42; HRMS(APCI): Calcd for C₆₂H₇₁N₇O₁₁S₃ [M−H]⁻: 1184.4301, found, 1184.4268.

### Example 19: Synthesis of Substrate V-1

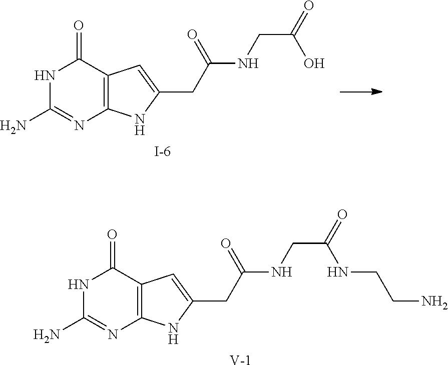

Substrate I-6 (530 mg, 2 mmol), benzotriazol-1-yl-oxytripyrrolidinophosphonium hexafluorophosphate (PyBOP, 1.2 g, 2.4 mmol), N-Boc-ethylenediamine (320 mg, 2 mmol), and N,N-diisopropylethylamine (516 mg, 4 mmol) were sequentially dissolved in anhydrous DMF (30 mL). The reaction mixture was stirred at room temperature for 4 hours. The solvent was removed under reduced pressure, and the crude product was purified by column chromatography. The purified intermediate (407 mg, 1 mmol) was dissolved in anhydrous dichloromethane (5 mL), followed by the addition of trifluoroacetic acid (5 mL). The mixture was stirred at room temperature for 3 hours. After removal of the solvent under reduced pressure, Substrate V-1 was obtained as a white solid (210 mg, 68.4% yield). ¹H NMR (600 MHz, DMSO-d₆) δ 8.26 (t, J=5.6 Hz, 1H), 8.19 (t, J=5.8 Hz, 1H), 6.14 (s, 2H), 6.00 (s, 1H), 3.68 (d, J=5.7 Hz, 2H), 3.42 (s, 2H), 3.18 (q, J=6.0 Hz, 2H), 2.69 (t, J=6.2 Hz, 2H); ¹³C NMR (151 MHz, DMSO-d₆) δ 174.18, 169.89, 169.56, 129.70, 125.15, 110.55, 102.76, 100.72, 100.32, 42.82, 38.39, 35.46, 23.46, 22.95; HRMS(APCI): Calcd for C₁₂H₁₇N₇O₃ [M+H]⁺: 308.1466, found 308.1478.

### Example 20: Synthesis of Substrate V-2

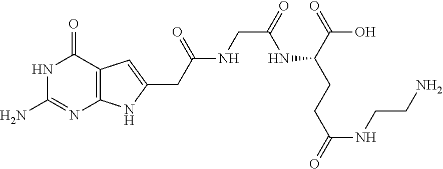 [SMILES: NCCNC(=O)CC[C@H](NC(=O)CNC(=O)Cc1cc2c(=O)[nH]c(N)nc2[nH]1)C(=O)O]

Following a procedure analogous to Example 19, Substrate V-2 was obtained (250 mg, 57.3% yield). ¹H NMR (600 MHz, DMSO-d₆) δ 10.90 (s, 1H), 8.31 (t, J=5.9 Hz, 1H), 8.02 (t, J=5.8 Hz, 1H), 7.60 (d, J=6.8 Hz, 1H), 6.20 (s, 2H), 6.03 (s, 1H), 3.86 (q, J=6.0 Hz, 1H), 3.69 (d, J=5.1 Hz, 2H), 3.45 (s, 2H), 3.33-3.15 (m, 2H), 2.84 (t, J=5.7, 4.6 Hz, 2H), 2.11-2.01 (m, 2H), 1.92-1.90 (m, 2H); ¹³C NMR (151 MHz, DMSO-d₆) δ 174.54, 173.14, 172.79, 169.99, 168.06, 159.13, 152.72, 151.72, 125.14, 100.80, 100.37, 53.70, 43.07, 37.66, 35.37, 31.97, 28.11, 22.04; HRMS(APCI): Calcd for C₁₇H₂₄N₈O₆ [M+H]⁺: 437.1892, found 437.1875.

### Example 21: Synthesis of Substrate V-3

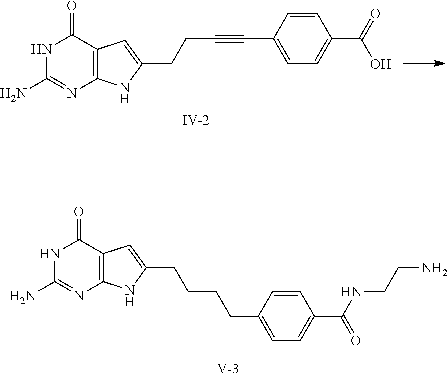

Substrate IV-2 (322 mg, 1 mmol) was dissolved in methanol (30 mL), and 5% palladium on carbon (100 mg) was added. The mixture was subjected to catalytic hydrogenation at 50 psi and room temperature for 12 hours. After filtration and removal of the solvent under reduced pressure, a white solid was obtained. Using a procedure analogous to Example 1, Substrate V-3 was isolated (300 mg, 81.5% yield). ¹H NMR (600 MHz, DMSO-d₆) δ 10.78 (s, 1H), 8.72 (t, J=5.5 Hz, 1H), 7.78 (d, J=7.8 Hz, 2H), 7.25 (d, J=7.8 Hz, 2H), 6.11 (s, 2H), 5.82 (s, 1H), 3.40 (q, J=6.0 Hz, 2H), 2.84 (t, 2H), 2.64 (t, J=6.4 Hz, 2H), 1.66-1.49 (m, 4H); ¹³C NMR (151 MHz, DMSO-d₆) δ 173.82, 166.98, 159.05, 152.50, 151.61, 146.15, 132.39, 131.41, 128.58, 127.80, 100.10, 98.26, 35.14, 30.76, 28.71, 27.45, 23.06; HRMS(APCI): Calcd for C₁₉H₂₄N₆O₂ [M+H]⁺: 369.2034, found 369.2039.

### Example 22: Synthesis of Substrate V-4

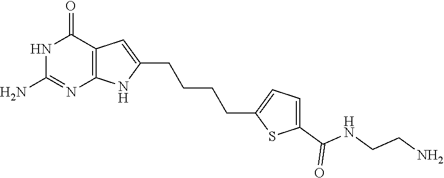 [SMILES: NCCNC(=O)c1ccc(CCCCc2cc3c(=O)[nH]c(N)nc3[nH]2)s1]

Following a procedure analogous to Example 21, Substrate V-4 was obtained (260 mg, 69.5% yield). ¹H NMR (600 MHz, DMSO-d₆) δ 10.79 (s, 1H), 8.83 (t, J=4.8 Hz, 1H), 7.59 (d, J=3.6 Hz, 1H), 6.85 (d, J=3.6 Hz, 1H), 6.14 (s, 2H), 5.84 (s, 1H), 3.34 (q, J=11.6, 5.8 Hz, 2H), 2.80 (t, J=13.1, 6.5 Hz, 4H), 1.65-1.55 (m, 4H); ¹³C NMR (151 MHz, DMSO-d₆) δ 173.82, 161.98, 159.07, 152.55, 151.63, 150.51, 137.62, 131.26, 128.73, 125.77, 100.09, 98.32, 31.03, 29.71, 28.47, 27.30, 23.15; HRMS(APCI): Calcd for C₁₇H₂₂N₆O₂S [M+H]+: 375.1598, found 375.1511.

### Example 23: Synthesis of Substrate V-5

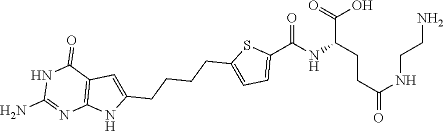 [SMILES: NCCNC(=O)CC[C@H](NC(=O)c1ccc(CCCCc2cc3c(=O)[nH]c(N)nc3[nH]2)s1)C(=O)O]

Following a procedure analogous to Example 19, Substrate V-5 was obtained (40 mg, 80%). ¹H NMR (600 MHz, DMSO-d₆) δ=10.81 (s, 1H), 8.34 (t, J=5.8 Hz, 1H), 7.91 (d, J=7.0 Hz, 1H), 7.56 (d, J=3.7 Hz, 1H), 6.82 (d, J=3.6 Hz, 1H), 6.16 (s, 2H), 5.86 (s, 1H), 4.07 (q, J=6.1 Hz, 2H), 3.37-3.14 (m, 2H), 2.84 (q, J=6.2 Hz, 2H), 2.78 (d, J=7.0 Hz, 2H), 2.15 (dt, J=13.7, 7.1 Hz, 2H), 2.00 (p, J=7.4, 7.0 Hz, 2H), 1.61 (dd, J=7.8, 3.9 Hz, 4H); ¹³C NMR (151 MHz, DMSO-d₆) δ=172.96, 171.19, 170.96, 158.50, 157.01, 150.42, 149.55, 148.07, 135.70, 129.23, 126.29, 123.65, 116.43, 97.96, 96.23, 52.09, 35.34, 30.15, 28.90, 27.59, 26.40, 26.08, 25.21, 20.10; HRMS(APCI): Calcd for C₂₂H₂₉N₇O₅S [M+H]⁺: 504.2018, found 504.2021.

### Example 24: Synthesis of Substrate V-6

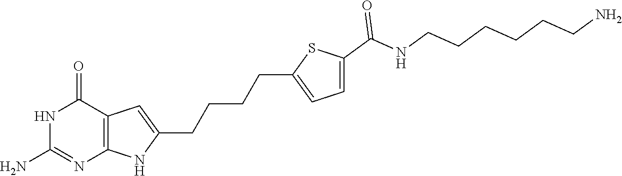 [SMILES: NCCCCCCNC(=O)c1ccc(CCCCc2cc3c(=O)[nH]c(N)nc3[nH]2)s1]

Following a procedure analogous to Example 19, reaction with N-(6-aminohexyl)carbamic acid tert-butyl ester afforded Substrate V-6 (32 mg, 75% yield). ¹H NMR (600 MHz, DMSO-d₆) δ=10.80 (s, 1H), 8.37 (t, J=5.7 Hz, 1H), 7.55 (d, J=3.7 Hz, 1H), 6.84 (d, J=3.7 Hz, 1H), 6.22 (s, 2H), 5.85 (s, 1H), 3.19 (q, J=6.6 Hz, 2H), 2.79 (d, J=6.9 Hz, 2H), 2.65 (t, J=7.4 Hz, 2H), 1.62 (p, J=3.7 Hz, 4H), 1.47 (dt, J=14.2, 7.1 Hz, 4H), 1.29 (dq, J=11.3, 5.7 Hz, 4H); ¹³C NMR (151 MHz, DMSO-d₆) δ=174.49, 161.51, 159.14, 152.64, 151.67, 150.24, 137.93, 131.23, 128.25, 125.69, 100.08, 98.32, 31.01, 29.84, 29.70, 29.56, 29.49, 28.50, 27.31, 26.58, 26.26, 24.19; HRMS(APCI): Calcd for C₂₁H₃₀N₆O₂S [M+H]⁺: 431.2224, found 431.2231.

### Example 25: Synthesis of Substrate V-7

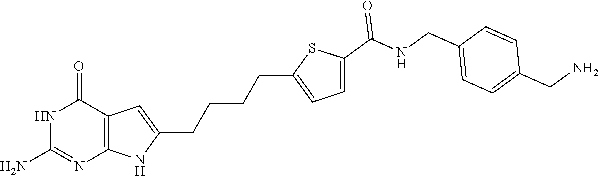 [SMILES: NCc1ccc(CNC(=O)c2ccc(CCCCc3cc4c(=O)[nH]c(N)nc4[nH]3)s2)cc1]

Following a procedure analogous to Example 19, reaction with N-[4-(aminomethyl)benzyl]carbamic acid tert-butyl ester afforded Substrate V-7 (34 mg, 75% yield). ¹H NMR (600 MHz, DMSO-d₆) δ=10.78 (s, 1H), 8.89 (t, J=6.1 Hz, 1H), 7.60 (d, J=3.7 Hz, 1H), 7.29 (d, J=8.1 Hz, 2H), 7.23 (d, J=8.1 Hz, 2H), 6.86 (d, J=3.7 Hz, 1H), 6.01 (s, 2H), 5.85 (d, J=2.1 Hz, 1H), 4.39 (d, J=6.0 Hz, 2H), 3.73 (s, 2H), 2.80 (t, J=6.7 Hz, 2H), 1.67-1.55 (m, 4H); ¹³C NMR (151 MHz, DMSO-d₆) δ=173.67, 161.62, 159.07, 152.52, 151.63, 150.68, 139.34, 138.87, 137.54, 131.29, 128.64, 128.18, 127.66, 125.84, 100.11, 98.36, 44.42, 42.66, 31.02, 29.72, 28.52, 27.32, 23.01; HRMS(APCI): Calcd for C₂₃H₂₆N₆O₂S [M+H]⁺: 451.1911, found 451.1906.

### Example 26: Synthesis of Substrate V-8

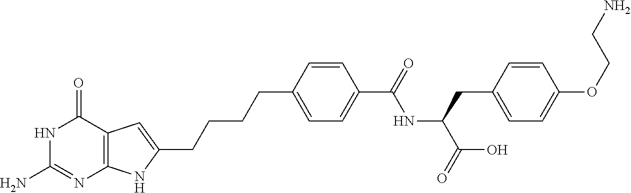 [SMILES: NCCOc1ccc(C[C@H](NC(=O)c2ccc(CCCCc3cc4c(=O)[nH]c(N)nc4[nH]3)cc2)C(=O)O)cc1]

Following a procedure analogous to Example 19, Substrate V-8 was obtained (41 mg, 77%). ¹H NMR (600 MHz, DMSO-d₆) δ=10.77 (s, 1H), 7.89-7.72 (m, 1H), 7.61 (d, J=6.8 Hz, 2H), 7.23 (d, J=7.9 Hz, 2H), 7.09 (d, J=8.2 Hz, 2H), 6.76 (d, J=7.2 Hz, 2H), 6.08 (s, 2H), 5.84 (d, J=2.1 Hz, 1H), 4.26 (q, J=5.9 Hz, 1H), 4.00 (dtd, J=15.4, 10.3, 5.3 Hz, 2H), 3.14 (dd, J=13.5, 5.0 Hz, 1H), 3.07 (t, J=5.6 Hz, 2H), 3.02 (dd, J=13.4, 6.6 Hz, 1H), 2.62 (p, J=4.0 Hz, 2H), 1.57 (q, J=3.7 Hz, 4H); ¹³C NMR (151 MHz, DMSO-d₆) δ=174.24, 172.71, 165.62, 159.12, 156.78, 152.57, 151.65, 145.97, 132.83, 131.83, 131.40, 130.85, 128.72, 127.35, 114.36, 100.08, 98.30, 65.74, 56.00, 36.49, 35.13, 30.73, 28.76, 27.49, 21.83; HRMS(APCI): Calcd for C₂₈H₃₂N₆O₅ [M+H]⁺: 533.2507, found 533.2518.

### Example 27: Synthesis of Substrate V-9

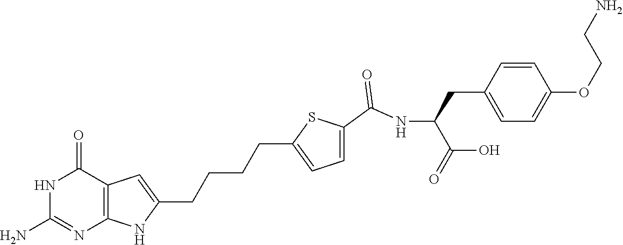 [SMILES: NCCOc1ccc(C[C@H](NC(=O)c2ccc(CCCCc3cc4c(=O)[nH]c(N)nc4[nH]3)s2)C(=O)O)cc1]

Following a procedure analogous to Example 19, Substrate V-9 was obtained (40 mg, 74%). ¹H NMR (600 MHz, DMSO-d₆) δ=10.80 (s, 1H), 7.83 (d, J=7.2 Hz, 1H), 7.45 (dd, J=3.7, 1.9 Hz, 1H), 7.10 (d, J=8.3 Hz, 2H), 6.79 (d, J=3.7 Hz, 1H), 6.76 (d, J=8.5 Hz, 2H), 6.20 (s, 2H), 5.85 (s, 1H), 4.25 (td, J=7.1, 4.8 Hz, 1H), 4.01 (hept, J=5.2 Hz, 2H), 3.10 (dt, J=14.0, 5.3 Hz, 3H), 2.96 (dd, J=13.5, 7.3 Hz, 1H), 2.77 (d, J=6.8 Hz, 2H), 1.60 (p, J=3.5 Hz, 4H); ¹³C NMR (151 MHz, DMSO-d₆) δ=174.17, 172.73, 160.66, 159.13, 156.81, 152.60, 151.67, 150.08, 137.82, 131.90, 131.28, 130.78, 128.29, 125.77, 114.40, 100.08, 98.34, 66.03, 56.14, 36.77, 31.01, 29.69, 28.53, 27.33, 21.87; HRMS(APCI): Calcd for C₂₆H₃₀N₆O₅S [M+H]+: 539.2071, found 539.2075.

### Example 28: Synthesis of Compound GT-FITC-1

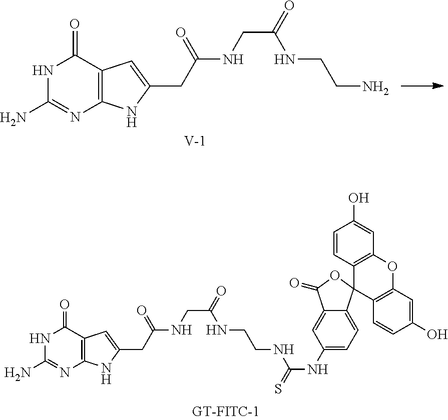

Substrate V-1 (31 mg, 0.1 mmol) was dissolved in DMSO (1 mL). To this solution, 5-isothiocyanatofluorescein (31 mg, 0.1 mmol) was added, followed by the addition of N,N-diisopropylethylamine (26 mg, 0.2 mmol). The reaction progress was monitored by HPLC. After completion, the crude product was purified by preparative liquid chromatography. The target fractions were lyophilized to afford GT-FITC-1 as an orange-yellow solid (21 mg, 30.1% yield). ¹H NMR (600 MHz, DMSO-d₆) δ 10.88 (s, 2H), 9.01 (s, 1H), 8.30 (s, 1H), 8.12 (t, J=15.3 Hz, 2H), 7.80 (dd, 1H), 7.15 (dd, J=8.3 Hz, 1H), 6.64 (d, J=2.2 Hz, 2H), 6.63 (d, J=8.7 Hz, 2H), 6.55 (dd, J=8.8, 2.3 Hz, 2H), 6.07 (s, 2H), 6.00 (s, 1H), 3.70 (d, J=5.5 Hz, 2H), 3.58 (q, 2H), 3.43 (s, 2H), 3.30 (q, J=6.1 Hz, 2H); ¹³C NMR (151 MHz, DMSO-d₆) δ 174.18, 173.55, 169.89, 169.56, 153.26, 151.81, 142.70, 129.69, 125.15, 110.54, 102.75, 100.71, 100.34, 100.29, 42.81, 40.52, 40.40, 38.39, 35.45, 23.43, 22.93; HRMS(APCI): Calcd for C₃₃H₂₈N₈O₈S [M+H]⁺: 697.1824, found 697.1811.

### Example 29: Synthesis of Compound GT-FITC-2

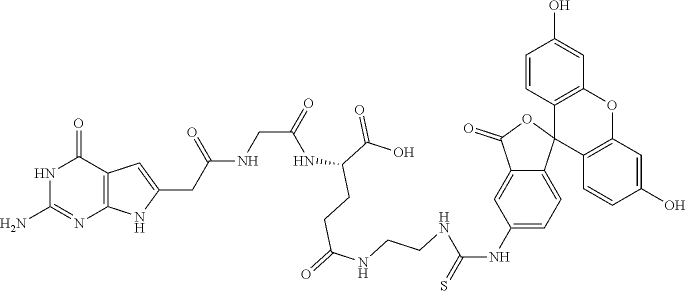 [SMILES: Nc1nc2[nH]c(CC(=O)NCC(=O)N[C@@H](CCC(=O)NCCNC(=S)Nc3ccc4c(c3)C(=O)OC43c4ccc(O)cc4Oc4cc(O)ccc43)C(=O)O)cc2c(=O)[nH]1]

Following a procedure analogous to Example 28, GT-FITC-2 was obtained as an orange-yellow solid (20 mg, 24.2% yield). ¹H NMR (600 MHz, DMSO-d₆) δ 12.04 (s, 1H), 10.82 (s, 1H), 9.96 (s, 1H), 8.50 (t, J=14.9 Hz, 1H), 8.15 (dt, J=30.1, 5.9 Hz, 1H), 7.95 (dd, J=13.6, 7.9 Hz, 1H), 7.77-7.65 (m, 1H), 7.62 (d, J=6.9 Hz, 1H), 7.12 (d, J=2.5 Hz, 1H), 6.66 (d, 2H), 6.61 (d, 2H), 6.56 (dd, J=8.6, 1.9 Hz, 2H), 6.14 (s, 1H), 6.07 (s, 1H), 6.01 (s, 1H), 3.96 (q, J=6.0 Hz, 1H), 3.69 (d, J=5.1 Hz, 2H), 3.59 (m, 2H), 3.45 (s, 2H), 3.23 (m, 2H), 2.14 (q, J=7.7, 7.2 Hz, 2H), 1.96 (dq, J=18.9, 9.8, 8.2 Hz, 2H); ¹³C NMR (151 MHz, DMSO-d₆) δ 181.05, 173.64, 172.69, 169.95, 169.38, 168.21, 159.17, 152.74, 151.70, 129.70, 110.72, 102.74, 100.74, 100.34, 54.22, 43.80, 42.92, 38.28, 35.38, 32.22, 22.94, 22.84; HRMS(APCI): Calcd for C₃₈H₃₅N₉O₁₁S [M+H]⁺: 826.2250 found 826.2251.

### Example 30: Synthesis of Compound GT-FITC-3

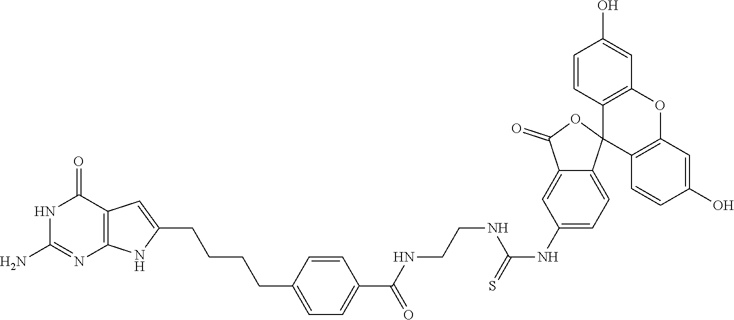 [SMILES: Nc1nc2[nH]c(CCCCc3ccc(C(=O)NCCNC(=S)Nc4ccc5c(c4)C(=O)OC54c5ccc(O)cc5Oc5cc(O)ccc54)cc3)cc2c(=O)[nH]1]

Following a procedure analogous to Example 28, GT-FITC-3 was obtained as an orange-yellow solid (25 mg, 33.1%). ¹H NMR (600 MHz, DMSO-d₆) δ 10.79 (d, J=2.3 Hz, 1H), 8.55 (t, J=6.5 Hz, 1H), 8.33 (t, 1H), 8.23 (s, 1H), 7.77 (d, J=7.9 Hz, 2H), 7.72 (s, 1H), 7.24 (d, J=7.9 Hz, 2H), 7.13 (d, J=8.3 Hz, 1H), 6.65 (d, 2H), 6.62 (d, J=8.7 Hz, 2H), 6.54 (dd, J=8.7, 2.4 Hz, 2H), 5.96 (s, 2H), 5.84 (s, 1H), 3.71 (q, 2H), 3.49 (q, J=6.0 Hz, 2H), 2.62 (t, J=6.7 Hz, 2H), 1.67-1.45 (m, 4H); ¹³C NMR (151 MHz, DMSO-d₆) δ 158.93, 152.31, 151.54, 146.21, 132.33, 131.51, 129.63, 128.62, 127.78, 102.74, 100.12, 98.30, 39.00, 35.15, 30.74, 28.79, 27.46; HRMS(APCI): Calcd for C₄₀H₃₅N₇O₇S [M+H]⁺: 758.2391, found 758.2381.

### Example 31: Synthesis of Compound GT-FITC-4

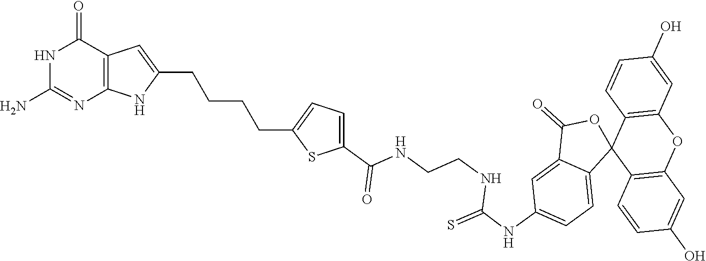 [SMILES: Nc1nc2[nH]c(CCCCc3ccc(C(=O)NCCNC(=S)Nc4ccc5c(c4)C(=O)OC54c5ccc(O)cc5Oc5cc(O)ccc54)s3)cc2c(=O)[nH]1]

Following a procedure analogous to Example 28, GT-FITC-4 was obtained as an orange-yellow solid (23 mg, 30.2%). ¹H NMR (600 MHz, DMSO-d₆) δ 10.80 (s, 1H), 10.15 (s, 2H), 8.55 (t, J=5.6 Hz, 1H), 8.23 (s, 2H), 7.74 (d, J=8.2 Hz, 1H), 7.59 (d, J=3.7 Hz, 1H), 7.15 (d, J=8.3 Hz, 1H), 6.84 (d, J=3.7 Hz, 1H), 6.68 (d, 2H), 6.61 (d, J=8.6 Hz, 2H), 6.56 (dd, J=8.7, 2.3 Hz, 2H), 5.98 (s, 2H), 5.86 (s, 1H), 3.69 (q, 2H), 3.46 (q, J=6.1 Hz, 2H), 2.80 (t, J=6.5 Hz, 2H), 1.65-1.49 (m, 4H); ¹³C NMR (151 MHz, DMSO-d₆) δ 168.98, 162.11, 160.00, 158.94, 152.37, 152.32, 151.57, 150.67, 147.73, 141.64, 137.47, 131.39, 129.52, 128.77, 125.80, 124.58, 113.08, 110.19, 102.72, 100.12, 98.36, 44.01, 38.79, 31.00, 29.71, 28.51, 27.29; HRMS(APCI): Calcd for C₃₈H₃₃N₇O₇S₂[M+H]⁺: 764.1956, found 764.1932.

### Example 32: Synthesis of Compound GT-FITC-5

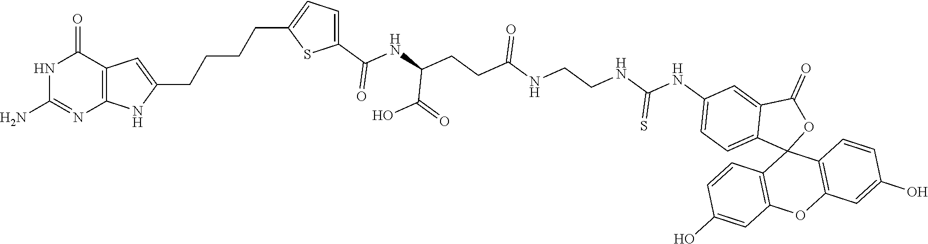 [SMILES: Nc1nc2[nH]c(CCCCc3ccc(C(=O)N[C@@H](CCC(=O)NCCNC(=S)Nc4ccc5c(c4)C(=O)OC54c5ccc(O)cc5Oc5cc(O)ccc54)C(=O)O)s3)cc2c(=O)[nH]1]

Following a procedure analogous to Example 28, GT-FITC-5 was obtained as an orange-yellow solid (16 mg, 36%). ¹H NMR (600 MHz, DMSO-d₆) δ=11.60 (s, 1H), 10.80 (s, 1H), 9.59 (s, 2H), 8.47 (s, 1H), 8.03-7.93 (m, 1H), 7.93-7.83 (m, 2H), 7.58 (s, 1H), 7.10 (d, J=8.2 Hz, 1H), 6.82 (s, 1H), 6.67 (s, 2H), 6.60 (d, J=8.7 Hz, 2H), 6.56 (d, J=9.1 Hz, 2H), 6.01 (s, 2H), 5.86 (s, 1H), 4.14 (d, J=7.5 Hz, 1H), 3.63 (d, J=11.4 Hz, 2H), 3.23 (s, 2H), 3.17 (s, 1H), 2.78 (s, 2H), 2.28-2.12 (m, 2H), 2.08 (d, J=13.0 Hz, 2H), 1.60 (s, 4H); ¹³C NMR (151 MHz, DMSO-d₆) δ=181.05, 175.50, 172.74, 169.24, 160.74, 160.11, 159.00, 152.45, 152.35, 151.61, 150.31, 142.91, 137.74, 131.43, 129.56, 128.50, 126.88, 125.75, 124.14, 116.35, 113.15, 110.42, 102.68, 100.12, 98.36, 54.25, 49.07, 43.68, 38.23, 32.29, 31.02, 29.71, 29.49, 29.05, 28.58, 27.80, 27.33, 21.75, 1.63; HRMS(APCI): Calcd for C₄₃H₃₉N₈O₁₀S₂ [M−H]⁻: 891.2236, found 891.2246.

### Example 33: Synthesis of Compound GT-FITC-6

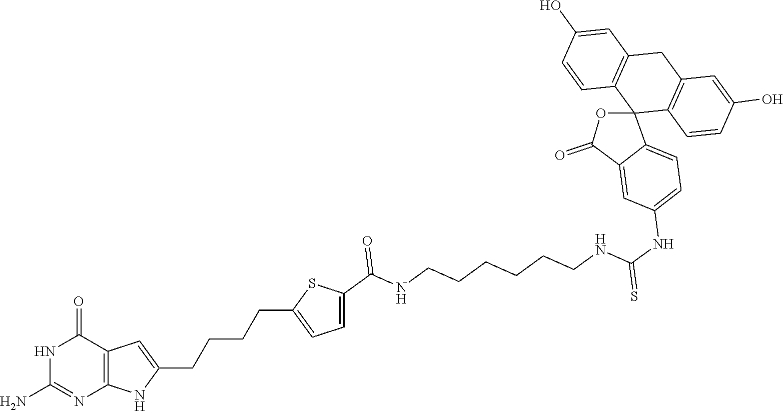 [SMILES: Nc1nc2[nH]c(CCCCc3ccc(C(=O)NCCCCCCNC(=S)Nc4ccc5c(c4)C(=O)OC54c5ccc(O)cc5Cc5cc(O)ccc54)s3)cc2c(=O)[nH]1]

Following a procedure analogous to Example 28, GT-FITC-6 was obtained as an orange-yellow solid (14 mg, 34%). ¹H NMR (600 MHz, DMSO-d₆) δ=10.81 (s, 1H), 10.12 (s, 2H), 8.34 (t, J=5.8 Hz, 1H), 8.31 (s, 1H), 8.26 (s, 1H), 7.74 (d, J=8.3 Hz, 1H), 7.55 (d, J=3.7 Hz, 1H), 7.15 (d, J=8.2 Hz, 1H), 6.83 (d, J=3.7 Hz, 1H), 6.65 (s, 2H), 6.63 (s, 1H), 6.56 (d, J=8.8 Hz, 2H), 5.98 (s, 2H), 5.87 (s, 1H), 3.51-3.47 (m, 2H), 3.21 (q, J=6.8 Hz, 2H), 3.18 (s, 1H), 2.79 (d, J=6.9 Hz, 2H), 1.62 (d, J=4.4 Hz, 4H), 1.54 (dt, J=28.2, 6.9 Hz, 4H), 1.34 (s, 4H); ¹³C NMR (151 MHz, DMSO-d₆) δ=180.82, 169.18, 161.52, 158.96, 152.82, 152.32, 151.57, 150.26, 141.89, 137.93, 131.40, 129.67, 128.24, 125.68, 110.62, 102.76, 100.13, 98.36, 49.07, 44.26, 31.01, 29.71, 29.68, 28.77, 28.53, 27.31, 26.70, 26.68; HRMS(APCI): Calcd for C₄₂H₄₀N₇O₇S₂ [M−H]⁻: 818.2436, found 818.2443.

### Example 34: Synthesis of Compound GT-FITC-7

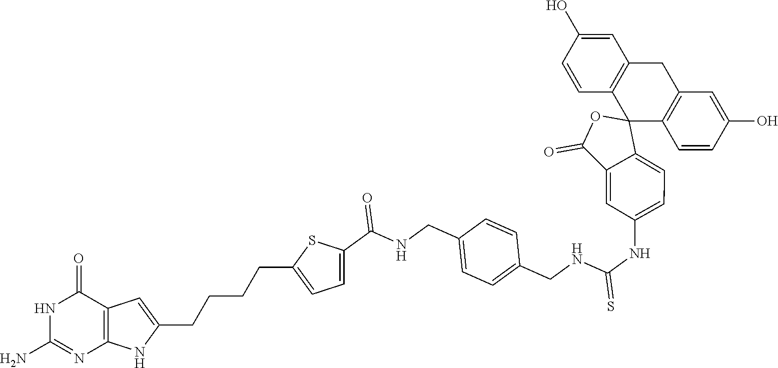 [SMILES: Nc1nc2[nH]c(CCCCc3ccc(C(=O)NCc4ccc(CNC(=S)Nc5ccc6c(c5)C(=O)OC65c6ccc(O)cc6Cc6cc(O)ccc65)cc4)s3)cc2c(=O)[nH]1]

Following a procedure analogous to Example 28, GT-FITC-7 was obtained as an orange-yellow solid (12 mg, 30%). ¹H NMR (600 MHz, DMSO-d₆) δ=10.79 (s, 1H), 10.14 (s, 2H), 8.90 (t, J=6.0 Hz, 1H), 8.58 (s, 1H), 8.26 (s, 1H), 7.75 (d, J=8.3 Hz, 1H), 7.60 (d, J=3.7 Hz, 1H), 7.32 (d, J=8.0 Hz, 2H), 7.28 (d, J=7.9 Hz, 2H), 7.17 (d, J=8.2 Hz, 1H), 6.86 (d, J=3.7 Hz, 1H), 6.64 (s, 1H), 6.62 (d, J=8.8 Hz, 2H), 6.57-6.52 (m, 2H), 5.96 (s, 2H), 5.85 (d, J=2.2 Hz, 1H), 4.80-4.72 (m, 2H), 4.41 (d, J=6.0 Hz, 2H), 2.80 (d, J=7.1 Hz, 2H), 1.65-1.58 (m, 4H); ¹³C NMR (151 MHz, DMSO-d₆) δ=161.60, 158.92, 152.32, 151.56, 150.68, 138.83, 137.53, 131.35, 129.62, 128.62, 127.93, 127.76, 125.83, 102.74, 100.13, 98.36, 42.68, 31.01, 29.72, 28.52, 27.30, 22.98, 22.56, 14.43; HRMS(APCI): Calcd for C₄₄H₃₆N₇O₇S₂[M−H]⁻: 838.2123, found 838.2122.

### Example 35: Synthesis of Compound GT-FITC-8

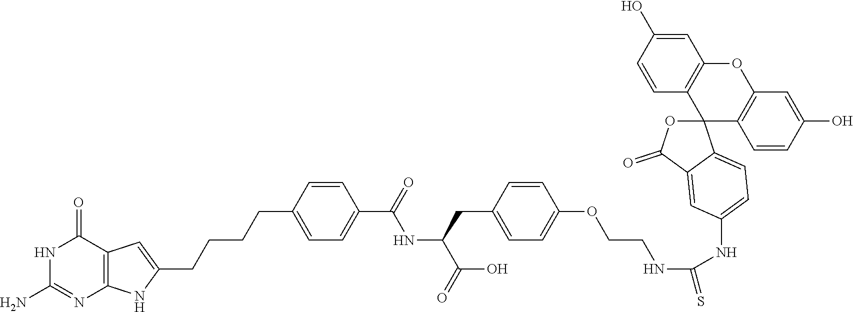 [SMILES: Nc1nc2[nH]c(CCCCc3ccc(C(=O)N[C@@H](Cc4ccc(OCCNC(=S)Nc5ccc6c(c5)C(=O)OC65c6ccc(O)cc6Oc6cc(O)ccc65)cc4)C(=O)O)cc3)cc2c(=O)[nH]1]

Following a procedure analogous to Example 28, GT-FITC-8 was obtained as an orange-yellow solid (15 mg, 32%). ¹H NMR (600 MHz, DMSO-d₆) δ=10.78 (s, 1H), 8.29 (s, 1H), 7.94 (s, 1H), 7.79 (d, J=8.3 Hz, 1H), 7.63 (d, J=7.8 Hz, 2H), 7.23 (d, J=7.9 Hz, 2H), 7.18 (d, J=8.3 Hz, 1H), 7.10 (d, J=8.1 Hz, 2H), 6.78 (d, J=8.0 Hz, 2H), 6.66 (d, J=2.1 Hz, 2H), 6.60 (s, 2H), 6.58 (s, 1H), 6.57 (dd, J=8.6, 2.2 Hz, 1H), 5.98 (s, 2H), 5.85 (d, J=2.2 Hz, 1H), 4.36 (d, J=6.7 Hz, 1H), 4.10-3.97 (m, 2H), 3.83 (s, 2H), 3.08 (dt, J=59.2, 7.6 Hz, 2H), 2.61 (t, J=6.3 Hz, 2H), 2.01 (m, 1H), 1.69-1.50 (m, 4H); ¹³C NMR (151 MHz, DMSO-d₆) δ=181.25, 174.49, 172.64, 169.05, 166.06, 160.16, 158.97, 157.16, 152.43, 152.34, 151.58, 146.12, 142.05, 132.55, 131.52, 131.33, 130.76, 130.12, 129.50, 128.67, 127.54, 124.49, 114.46, 113.16, 110.22, 110.19, 102.71, 100.12, 98.31, 65.91, 55.67, 43.77, 36.39, 35.59, 35.16, 31.74, 30.76, 29.50, 29.29, 29.19, 29.16, 29.05, 29.03, 28.85, 27.49, 27.03, 22.98, 22.56, 21.67, 14.43; HRMS(APCI): Calcd for C₄₉H₄₂N₇O₁₀S [M−H]⁻: 920.2719, found 920.2716.

### Example 36: Synthesis of Compound GT-FITC-9

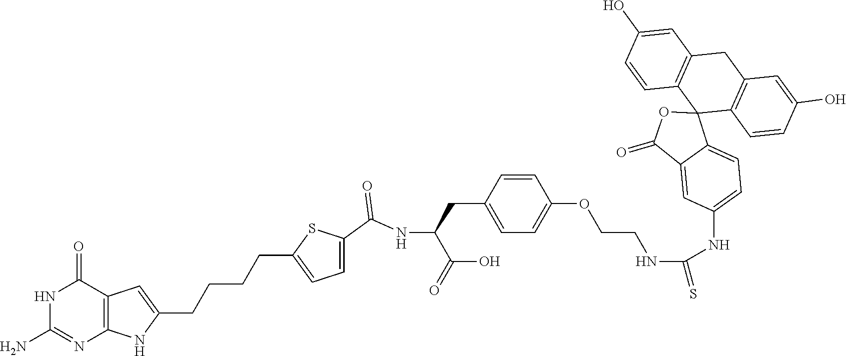 [SMILES: Nc1nc2[nH]c(CCCCc3ccc(C(=O)N[C@@H](Cc4ccc(OCCNC(=S)Nc5ccc6c(c5)C(=O)OC65c6ccc(O)cc6Cc6cc(O)ccc65)cc4)C(=O)O)s3)cc2c(=O)[nH]1]

Following a procedure analogous to Example 28, GT-FITC-9 was obtained as an orange-yellow solid (16 mg, 35%). ¹H NMR (600 MHz, DMSO-d₆) δ=10.78 (s, 1H), 8.69 (s, 1H), 8.29 (s, 1H), 8.16 (s, 1H), 7.77 (d, J=8.2 Hz, 1H), 7.52 (d, J=3.6 Hz, 1H), 7.17 (d, J=8.3 Hz, 1H), 7.14 (d, J=8.1 Hz, 3H), 6.83 (s, 2H), 6.82 (d, J=4.6 Hz, 2H), 6.68-6.65 (m, 3H), 5.97 (s, 2H), 5.85 (s, 1H), 4.37 (p, J=10.6, 7.1 Hz, 1H), 4.08 (tt, J=10.2, 5.0 Hz, 2H), 3.85 (s, 2H), 3.09 (dd, J=13.7, 4.6 Hz, 1H), 2.96 (dd, J=13.8, 8.5 Hz, 1H), 2.78 (d, J=6.3 Hz, 2H), 1.61 (d, J=6.0 Hz, 4H); ¹³C NMR (151 MHz, DMSO-d₆) δ=181.26, 174.78, 174.46, 169.04, 161.16, 160.09, 158.97, 157.20, 152.40, 152.35, 151.60, 150.51, 142.00, 137.48, 131.38, 131.28, 130.69, 130.13, 129.50, 128.66, 127.05, 125.76, 124.48, 117.02, 114.54, 113.13, 110.21, 102.71, 100.12, 98.36, 65.94, 55.58, 43.77, 36.49, 35.60, 31.76, 31.02, 29.71, 29.56, 29.50, 29.45, 29.34, 29.30, 29.21, 29.17, 29.05, 28.58, 27.33, 27.03, 25.59, 22.57, 21.65, 14.42, 1.63; HRMS(APCI): Calcd for C₄₇H₄₂N₇O₁₀S₂ [M+H]⁺: 928.2429, found 928.2420.

### Example 37: Synthesis of Compound GT-IR-1

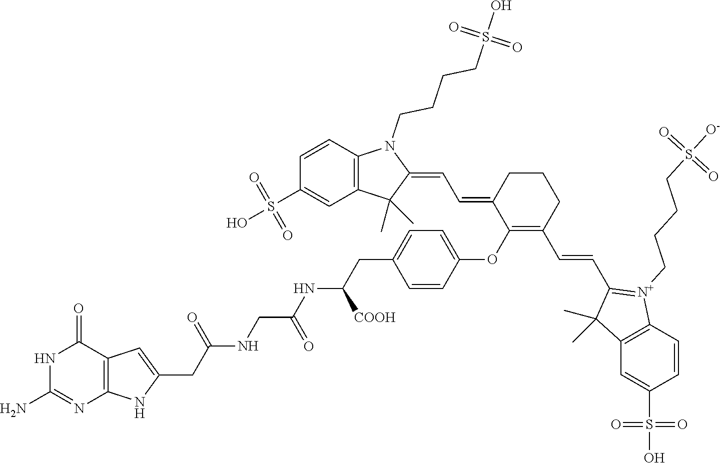 [SMILES: CC1(C)C(/C=C/C2=C(Oc3ccc(C[C@H](NC(=O)CNC(=O)Cc4cc5c(=O)[nH]c(N)nc5[nH]4)C(=O)O)cc3)C(=C/C=C3/N(CCCCS(=O)(=O)O)c4ccc(S(=O)(=O)O)cc4C3(C)C)/CCC2)=[N+](CCCCS(=O)(=O)[O-])c2ccc(S(=O)(=O)O)cc21]

Following a procedure analogous to that described in Example 14, GT-IR-1 was obtained as a dark green solid (33 mg, 29%). ¹H NMR (600 MHz, DMSO-d₆) δ 10.83 (s, 1H), 8.17 (s, 1H), 7.81 (d, J=14.0 Hz, 2H), 7.53 (d, J=7.6 Hz, 2H), 7.38 (t, J=8.5 Hz, 3H), 7.32 (d, J=7.7 Hz, 2H), 7.16 (d, J=8.4 Hz, 2H), 6.96 (d, J=8.4 Hz, 2H), 6.26 (t, J=19.0 Hz, 2H), 6.18 (d, J=14.0 Hz, 2H), 5.95 (s, 1H), 4.14 (t, J=7.4 Hz, 4H), 4.05-4.00 (m, 1H), 3.59 (ddd, J=58.4, 17.8, 6.8 Hz, 2H), 3.37-3.27 (m, 2H), 3.03-2.80 (m, 2H), 2.72 (t, J=6.5 Hz, 4H), 1.78-1.66 (m, 10H), 1.34 (dd, J=15.6, 4.7 Hz, 12H); ¹³C NMR (151 MHz, DMSO-d₆) δ 174.92, 172.68, 169.71, 162.23, 159.15, 157.53, 152.65, 150.66, 142.52, 140.45, 132.81, 132.05, 131.52, 130.12, 128.87, 125.14, 114.43, 111.67, 100.67, 100.36, 51.19, 48.99, 43.98, 35.58, 35.24, 32.75, 29.55, 29.49, 29.46, 29.30, 29.21, 29.16, 29.04, 27.74, 27.69, 27.22, 26.47, 25.48, 24.15, 22.93, 22.56, 21.52; HRMS(APCI): Calcd for C₅₇H₆₆N₈O₁₈S₄ [M−H]⁻: 1277.3305, found, 1277.3314.

### Example 38: Synthesis of Compound GT-IR-2

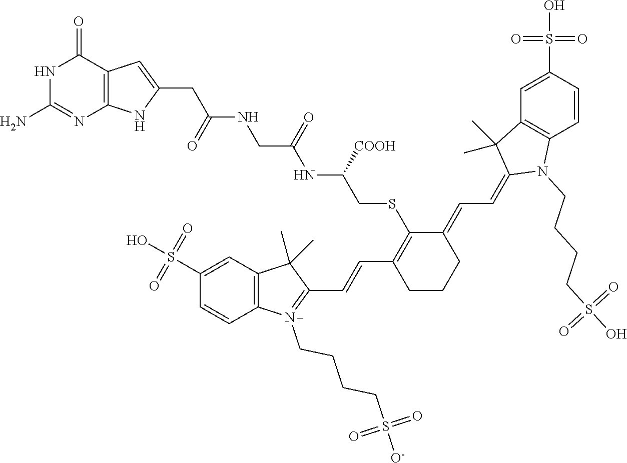

Following a procedure analogous to that described in Example 14, GT-IR-2 was obtained as a dark green solid (23 mg, 373%). H NMR (600 MHz, DMSO-d₆) δ 10.88 (s, 1H), 8.47 (d, J 13.9 Hz, 2H), 8.21 (d, J=5.7 Hz, 1H), 7.73 (d, J=6.2 Hz, 1H), 7.51 (d, J=7.4 Hz, 2H), 7.44 (d, J=8.0 Hz, 2H), 7.34 (t), J=7.6 Hz, 2H), 6.36 (d, J 14.2 Hz, 2H), 6.33 (s, 2H), 6.03 (s, 1H), 4.26 (t, J=7.7 Hz, 4H), 4.02 (q, J=5.6 Hz, 1H), 3.69 (qd, J=16.7, 5.6 Hz, 2H), 3.47 (d, J=3.0 Hz, 2H), 3.34-3.11 (m, 2H), 2.51 (t, J=7.4 Hz, 4H), 1.71 (q, J=7.4 Hz, 4H), 1.64 (hept, J=6.5, 6.1 Hz, 6H), 1.57 (s, 12H); ¹³C NMR (151 MHz, DMSO-d₆) δ 174.70, 172.99, 167.83, 168.51, 159.41, 158.59, 152.72, 151.56, 145.78, 142.71, 141.49, 135.34, 133.09, 132.83, 128.93, 125.19, 111.68, 101.58, 100.74, 100.33, 55.48, 51.28, 49.28, 43.94, 42.85, 35.36, 28.11, 27.92, 26.45, 26.18, 22.99, 21.59; HRMS(APCI): Calcd for C₅₁H₆₂N₈O₁₇S₅[M−H]⁻: 1217.2763, found, 1217.2760.

### Example 39: Synthesis of Compound GT-IR-3

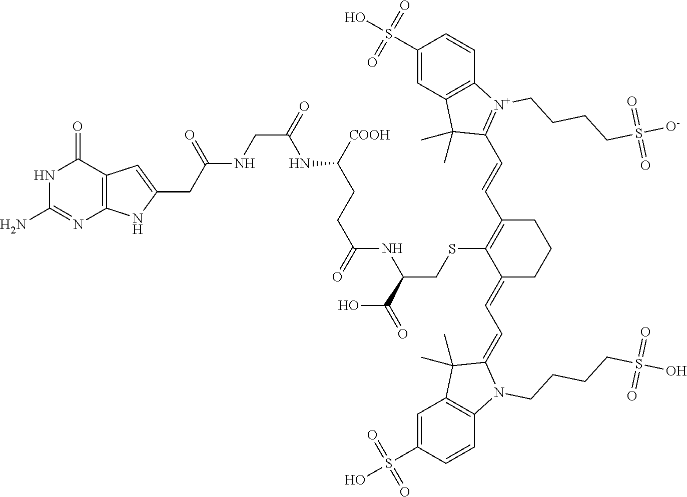 [SMILES: CC1(C)C(/C=C/C2=C(SC[C@H](NC(=O)CC[C@H](NC(=O)CNC(=O)Cc3cc4c(=O)[nH]c(N)nc4[nH]3)C(=O)O)C(=O)O)C(=C/C=C3/N(CCCCS(=O)(=O)O)c4ccc(S(=O)(=O)O)cc4C3(C)C)/CCC2)=[N+](CCCCS(=O)(=O)[O-])c2ccc(S(=O)(=O)O)cc21]

Following a procedure analogous to that described in Example 14, GT-IR-3 was obtained as a dark green solid (22 mg, 34%). ¹H NMR (600 MHz, DMSO-d₆) δ 11.02 (s, 1H), 8.57 (d, J=13.7 Hz, 2H), 8.26 (t, J=6.0 Hz, 1H), 7.66 (d, J=6.9 Hz, 1H), 7.56 (d, J=7.3 Hz, 2H), 7.54 (d, J=6.3 Hz, 1H), 7.37 (d, J=8.1 Hz, 2H), 7.31 (t, J=7.5 Hz, 2H), 6.44 (s, 2H), 6.26 (d, J=13.9 Hz, 2H), 6.12 (s, 1H), 4.16 (d, J=8.8 Hz, 4H), 4.01 (d, J=6.8 Hz, 1H), 3.93 (q, J=6.4 Hz, 1H), 3.71-3.58 (m, 2H), 3.48 (s, 2H), 3.33-3.12 (m, 2H), 2.56 (t, J=7.4 Hz, 5H), 2.16-2.03 (m, 2H), 1.95-1.89 (m, 2H), 1.85 (q, J=7.4 Hz, 4H), 1.72 (q, J=7.8, 7.2 Hz, 6H), 1.67 (s, 12H); ¹³C NMR (151 MHz, DMSO-d₆) δ 174.81, 172.88, 172.74, 172.26, 171.95, 170.17, 167.51, 159.28, 152.71, 151.76, 144.19, 143.73, 141.58, 135.26, 135.05, 133.22, 128.79, 122.42, 112.58, 101.57, 100.86, 100.29, 55.47, 53.96, 51.22, 49.28, 43.96, 43.04, 41.21, 35.35, 32.81, 28.88, 28.07, 27.92, 26.44, 25.17, 22.86, 22.21, 20.78; HRMS(APCI): Calcd for C₅₆H₆₉N₉O₂₀S₅[M−H]⁻: 1346.3190, found, 1346.3199.

### Example 40: Synthesis of Compound GT-IR-4

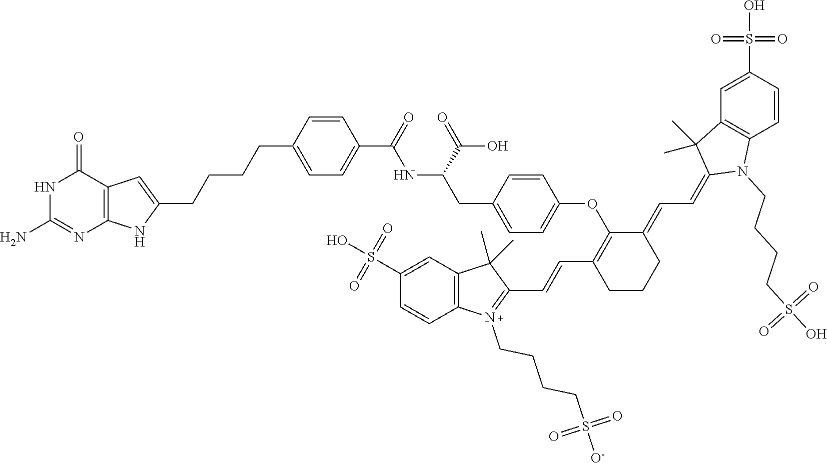

Following a procedure analogous to that described in Example 14, GT-IR-4 was obtained as a dark green solid (26 mg, 28.3%). ¹H NMR (600 MHz, DMSO-d₆) δ 10.88 (s, 1H), 7.98 (s, 1H), 7.71 (d, J=13.9 Hz, 2H), 7.59 (d, J=7.4 Hz, 2H), 7.39 (s, 2H), 7.28 (d, J=8.0 Hz, 4H), 7.10 (t, J=7.3 Hz, 2H), 7.04 (d, J=7.8 Hz, 2H), 6.98 (d, J=7.8 Hz, 2H), 6.24 (s, 2H), 6.35 (d, J=14.1 Hz, 2H), 5.73 (s, 1H), 4.29 (s, 1H), 4.08 (s, 4H), 3.24-2.98 (m, 2H), 2.67 (s, 4H), 2.57-2.52 (m, 5H), 1.77 (d, J=23.7 Hz, 8H), 1.61-1.51 (m, 2H), 1.46 (q, J=7.7 Hz, 2H), 1.26-1.20 (m, 2H), 1.14 (s, 12H); ¹³C NMR (151 MHz, DMSO-d₆) δ 174.09, 173.86, 166.87, 163.30, 158.33, 153.58, 151.70, 145.89, 143.42, 141.46, 141.28, 134.69, 134.06, 133.52, 132.74, 131.45, 128.82, 128.44, 127.51, 125.75, 114.21, 112.67, 100.57, 100.25, 98.22, 50.18, 48.83, 43.98, 41.39, 40.18, 39.91, 39.84, 39.53, 36.68, 35.10, 30.68, 29.41, 28.72, 27.69, 27.49, 26.43, 25.93, 24.16, 22.99, 22.98, 22.83, 22.51, 22.26, 21.49; HRMS(APCI): Calcd for C₆₄H₇₃N₇O₁₇S₄[M−H]⁻: 1338.3873, found, 1338.3889.

### Example 41: Synthesis of Compound GT-IR-5

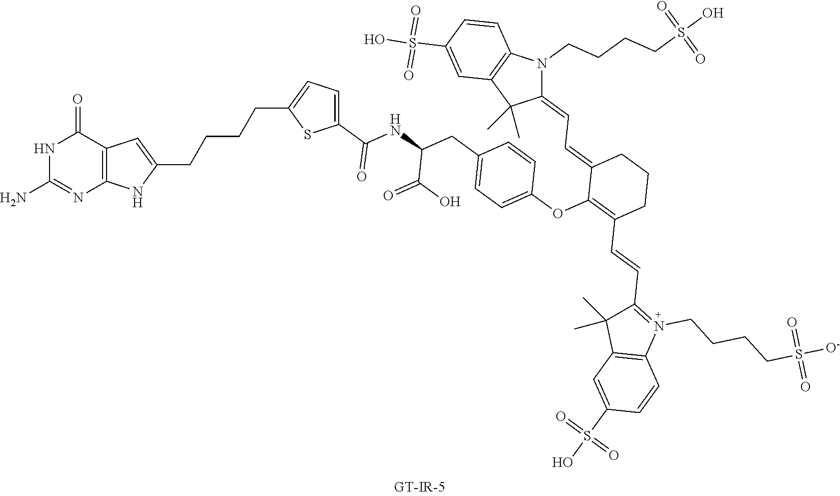

Following a procedure analogous to that described in Example 14, GT-IR-5 was obtained as a dark green solid (24 mg, 25%). ¹H NMR (600 MHz, DMSO-d₆) δ 10.89 (s, 1H), 8.39-8.29 (m, 1H), 7.73 (d, J=13.9 Hz, 2H), 7.59 (d, J=3.9 Hz, 1H), 7.42-7.20 (m, 8H), 7.08 (d, J=8.0 Hz, 2H), 6.75 (d, J=3.9 Hz, 1H), 6.12 (d, J=14.1 Hz, 2H), 6.03 (s, 2H), 5.82 (s, 1H), 4.33 (t, J=10.3 Hz, 1H), 4.24 (s, 4H), 3.15-2.89 (m, 2H), 2.69 (s, 4H), 2.65-2.57 (m, 2H), 2.44 (s, 4H), 1.71 (d, J=17.2 Hz, 8H), 1.57 (p, J=7.1 Hz, 2H), 1.47 (q, J=7.7 Hz, 2H), 1.26 (s, 4H), 1.12 (s, 12H); ¹³C NMR (151 MHz, DMSO-d₆) δ 173.58, 171.89, 164.21, 161.37, 159.01, 157.49, 152.43, 150.46, 142.77, 141.51, 137.33, 132.92, 131.34, 130.12, 128.84, 128.68, 125.34, 125.05, 114.41, 111.62, 100.68, 98.35, 55.58, 51.12, 49.37, 48.85, 36.14, 35.51, 31.73, 30.77, 29.42, 29.16, 28.58, 27.69, 27.02, 26.41, 24.82, 24.41, 22.99, 22.54, 21.68, 21.24; HRMS(APCI): Calcd for C₆₂H₇₁N₇O₁₇S₅[M−H]⁻: 1344.3437, found, 1344.3451.

### Example 42: Determination of Optical Properties

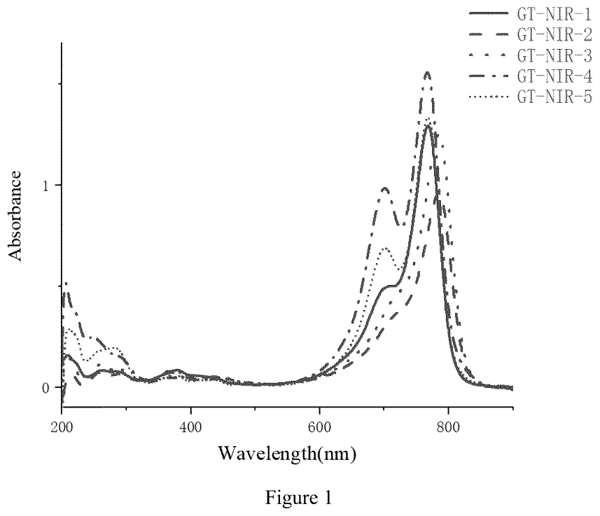

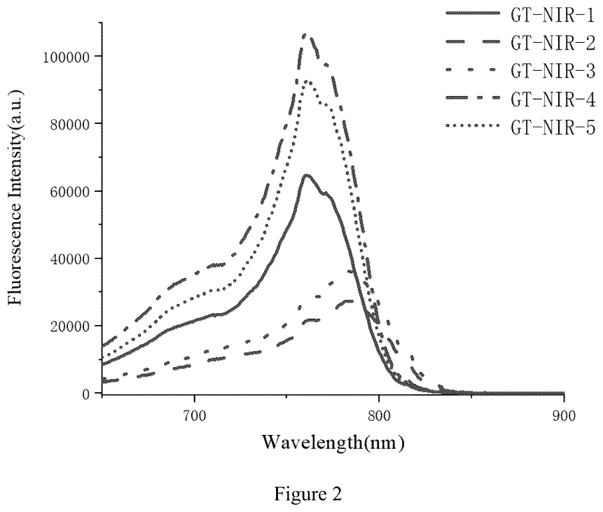

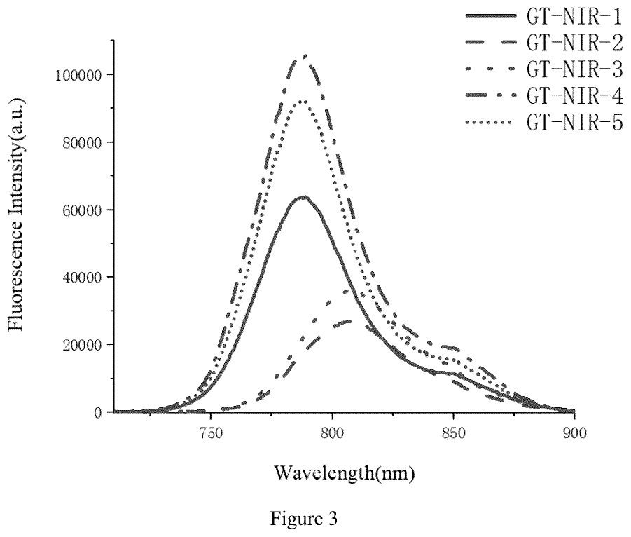

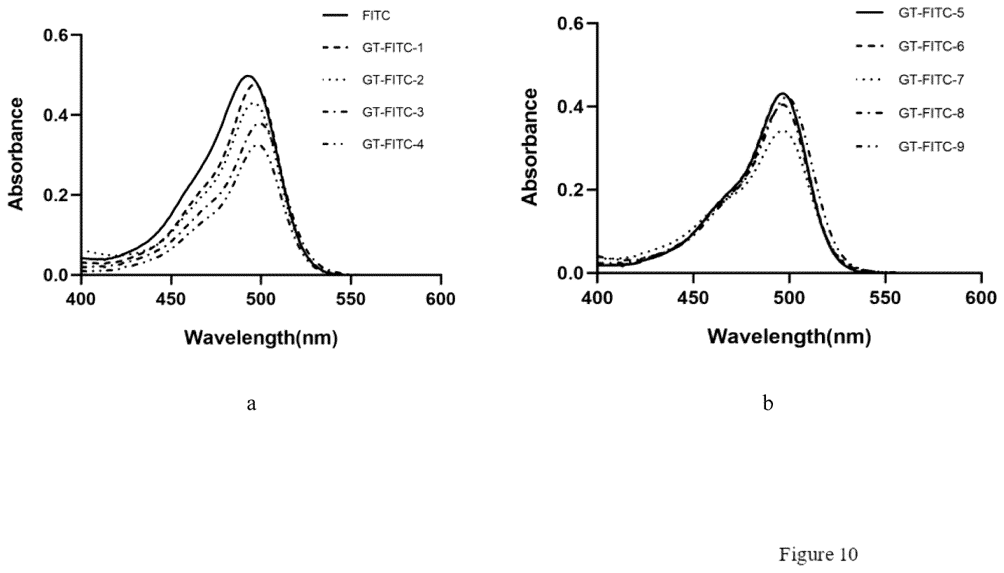

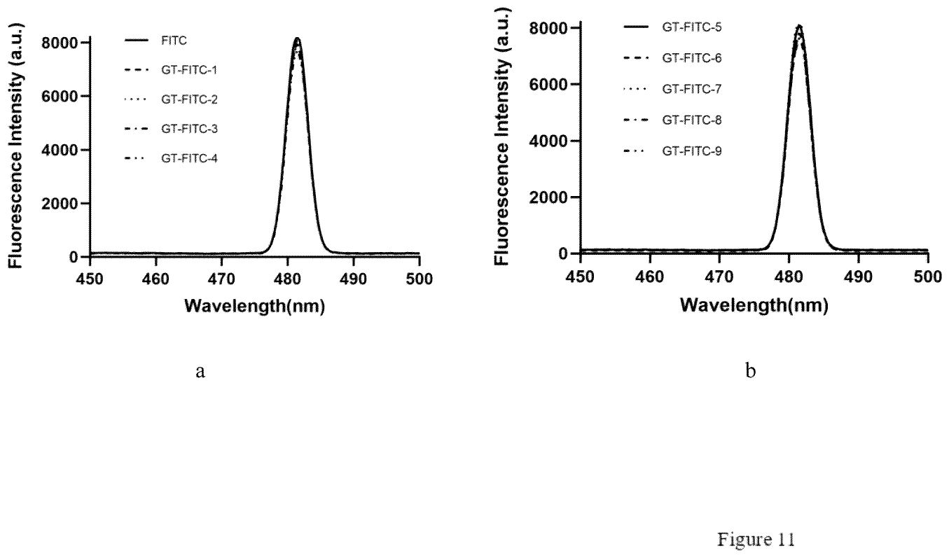

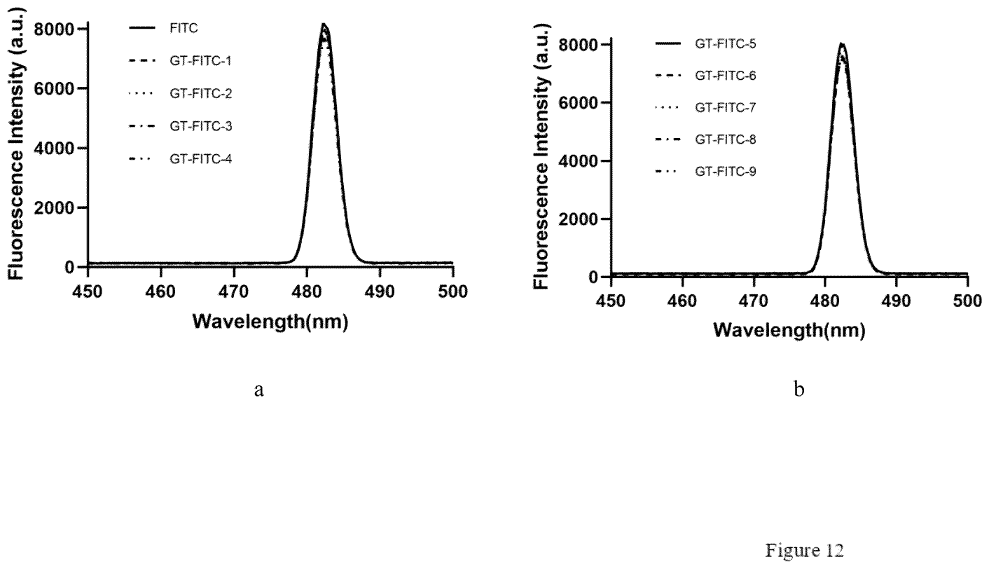

The absorption spectra of all compounds (10 μM) were measured in PBS solution using a T9S UV-Vis spectrophotometer across the wavelength range of 200-900 nm. The fluorescence spectra of the GT-NIR series compounds (1 μM) and GT-FITC series compounds (100 nM) were recorded in PBS solution using an RF-6000 fluorescence spectrophotometer. The results are presented in FIGS. 1-3 and 10-12. Both the GT-NIR series and GT-FITC series probe compounds exhibited optical properties similar to their respective parent fluorophores with minimal spectral shifts.

### Example 43: In Vitro Fluorescence Microscopy Assay

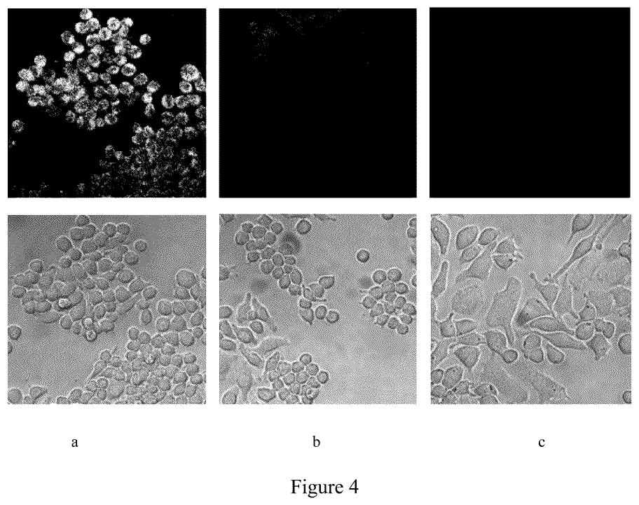

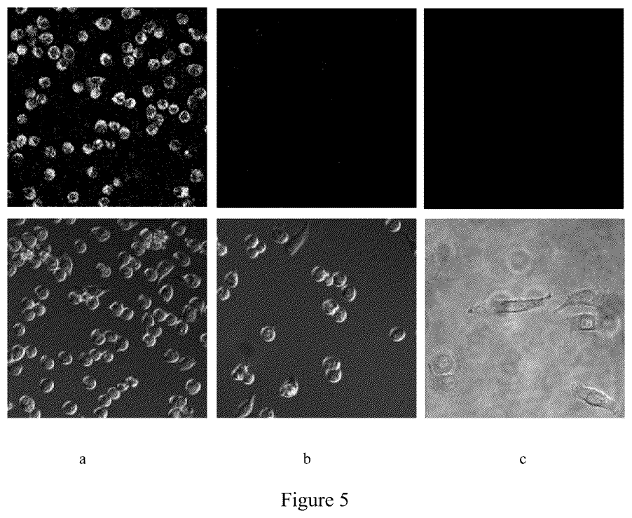

To investigate the targeting capability of compound GT-NIR-1 toward folate receptor-expressing cells and its subcellular localization, FR⁺ KB cells, FR⁻ A549 cells, FR⁺ M2-type macrophages, and FR⁻ M1-type macrophages were seeded in confocal-specific culture dishes at a density of 3×10⁵ cells per dish. The cells were cultured at 37° C. under 5% CO₂ for 24 hours. Subsequently, the cells were incubated with GT-NIR-1 (1 μM) for 30 minutes. For competitive binding assays, KB cells and M2 macrophages were co-treated with a 100-fold excess of folic acid (100 μM) and GT-NIR-1 (1 μM) for 30 minutes. After incubation, all cells were washed three times with PBS (pH=7.4). Fluorescence was excited at 775 nm, and cellular fluorescence imaging was observed using confocal fluorescence microscopy (FIG. 4 and FIG. 5). The results demonstrated that probe GT-NIR-1 successfully entered cells via targeting folate receptors, including FRα (in KB cells) and FRβ (in M2 macrophages), while no accumulation was observed in folate receptor-negative cells (A549 cells and M1 macrophages). This indicates that probe GT-NIR-1 possesses the ability to target tumor cells with high folate receptor expression and enable fluorescence imaging.

### Example 44: In Vitro Affinity Assay of GT-NIR Series Probes

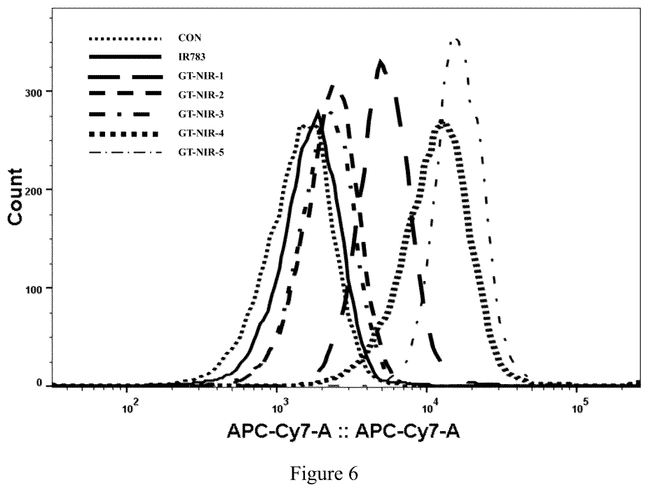

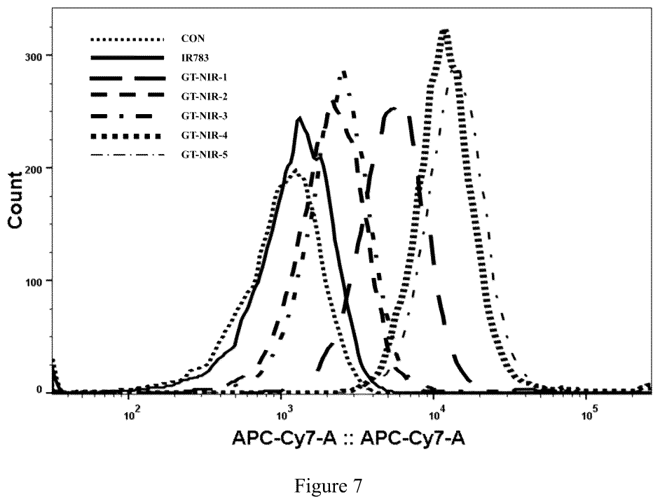

To compare the targeting affinity of different compounds in the GT-NIR series for folate receptors, FR⁺ KB cells and M2 macrophages were seeded in 6-well plates at a density of 1×10⁶ cells per well. After 24 hours of culture, the medium was replaced with folate-free fresh medium as a blank control. IR783 (500 nM) was added to exclude false-positive effects. Media containing GT-NIR series compounds (500 nM) were added to different wells and incubated for 1 hour. The cells were then digested with trypsin, washed three times with PBS (pH=7.4), and centrifuged (2000 rpm, 5 minutes) to remove unbound probes. The cells were resuspended in 500 mL PBS, stored in light-protected BD Falcon tubes, and analyzed by flow cytometry. Statistical data were processed using FlowJo software (FIG. 6 and FIG. 7). The results indicated that GT-NIR-5 exhibited the highest binding affinity for both FRα and FRβ, followed by GT-NIR-4 and GT-NIR-1.

### Example 45: In Vitro Affinity Assay of GT-FITC Series Probes

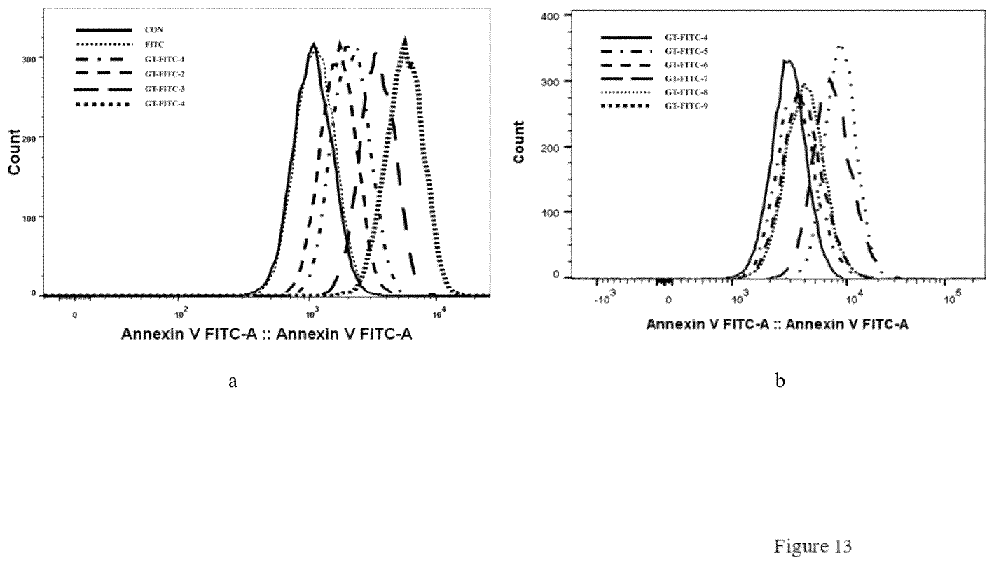

To evaluate the targeting affinity of different compounds in the GT-FITC series for folate receptors, KB cells were seeded in 6-well plates at a density of 1×10⁶ cells per well. After 24 hours of culture, the medium was replaced with folate-free fresh medium as a blank control. FITC (500 nM) was added to exclude false-positive interference. Media containing GT-FITC series compounds (500 nM) were added to different wells and incubated for 1 hour. The cells were digested with trypsin, washed three times with PBS (pH=7.4), and centrifuged (2000 rpm, 5 minutes) to remove unbound probes. The cells were resuspended in 500 mL PBS, stored in light-protected BD Falcon tubes, and analyzed by flow cytometry. Statistical data were processed using FlowJo software (FIG. 13). The results in FIG. 13(a) showed that among compounds with different targeting ligands, GT-FITC-4 had the highest binding affinity for KB cells, followed by GT-FITC-3 and GT-FITC-1. The results in FIG. 13(b) indicated that for compounds with the same targeting ligand, although other linkers did not reduce (and even slightly enhanced) the affinity for folate receptors, tyrosine-based linkers performed the best. These results demonstrate that all GT-FITC series probes can effectively target and enter tumor cells with high folate receptor expression.

### Example 46: Determination of Equilibrium Dissociation Constant (K_{d})

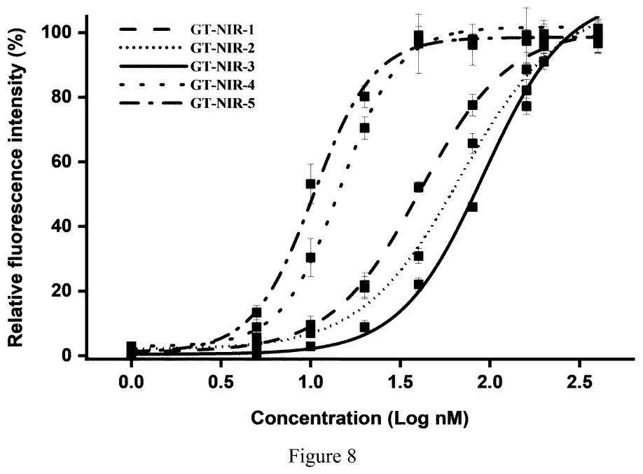

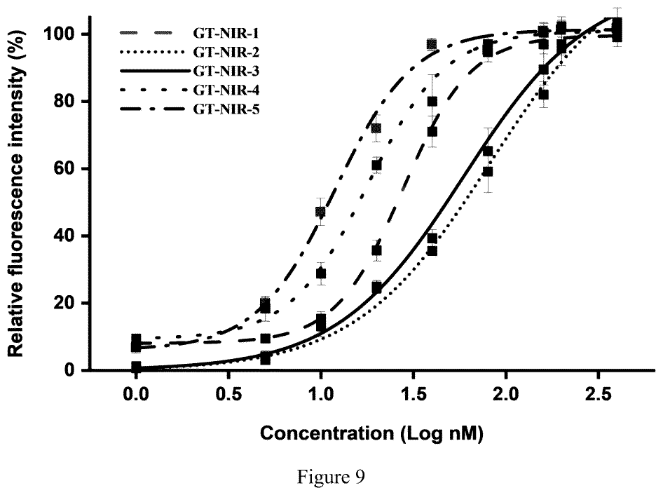

The GT-NIR series compounds were sequentially diluted in fresh medium to concentrations ranging from 1 nM to 400 nM. These solutions were incubated with 10⁶ KB cells and M2-type macrophages at 4° C. for 0.5 hours. After incubation, the cells were washed three times with cold PBS (pH=7.4) to remove unbound probes. The cells were then resuspended in 500 mL PBS, stored in light-protected BD Falcon tubes, and analyzed by flow cytometry to quantify fluorescence intensity. The equilibrium dissociation constant K_{d} was calculated by plotting a nonlinear regression curve using GraphPad Prism 8.0 software. The results are shown in FIG. 8 and FIG. 9.

### Example 47: Quantitative Analysis of In Vitro Cellular Fluorescence

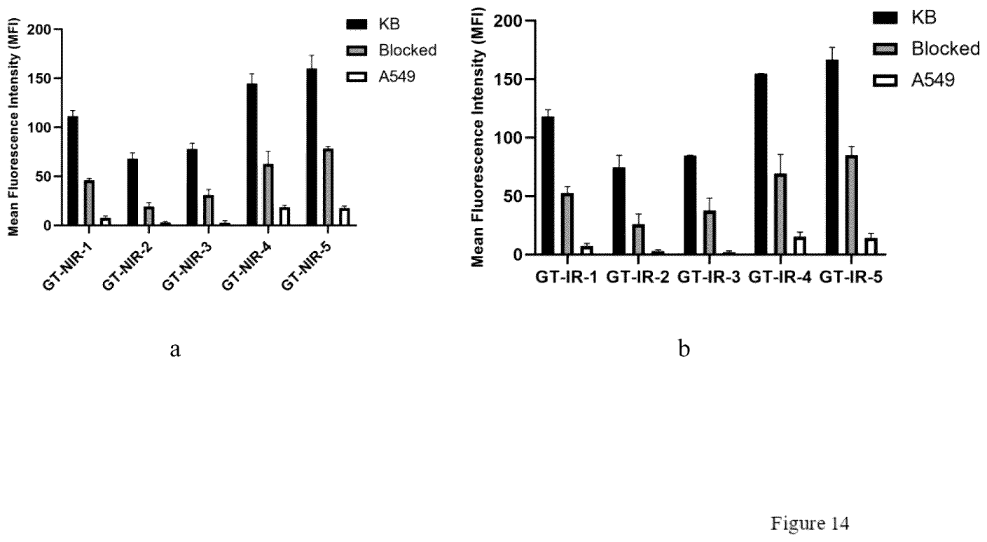

It is well established that the selectivity of targeted fluorescent probes is determined by the targeting ligand moiety. Since the GT-NIR series compounds and GT-IR series compounds differ only in their fluorophore components, it was hypothesized that both series would exhibit similar capabilities for targeting tumor cells with high folate receptor (FR) expression. Following the same experimental procedure as described in Example 43, a quantitative fluorescence analysis was performed for all compounds in the GT-NIR and GT-IR series. The results are presented in FIG. 14(a) and FIG. 14(b). The data demonstrate that both GT-NIR and GT-IR series compounds can target and enter tumor cells with high FR expression, enabling fluorescence imaging in the near-infrared region.

### Example 48: In Vivo Small Animal Imaging Studies

Subcutaneous tumor models were established using FR⁺ KB cell xenografts and FR A549 cell xenografts. The A549 xenograft model served as the negative control group, while a competition group was included to validate the in vivo targeting specificity of the probes toward tissues with high folate receptor expression. When tumor volumes reached 300-400 mm³, the following treatments were administered via tail vein injection:
Blank control group: 100 μL saline;
Experimental groups: respective probes (10 nmol, dissolved in 100 μL saline);
Competition group: pre-injection of 100× excess folic acid (1 mol in 100 μL saline) 15 minutes prior to probe administration (10 nmol in 100 μL saline).

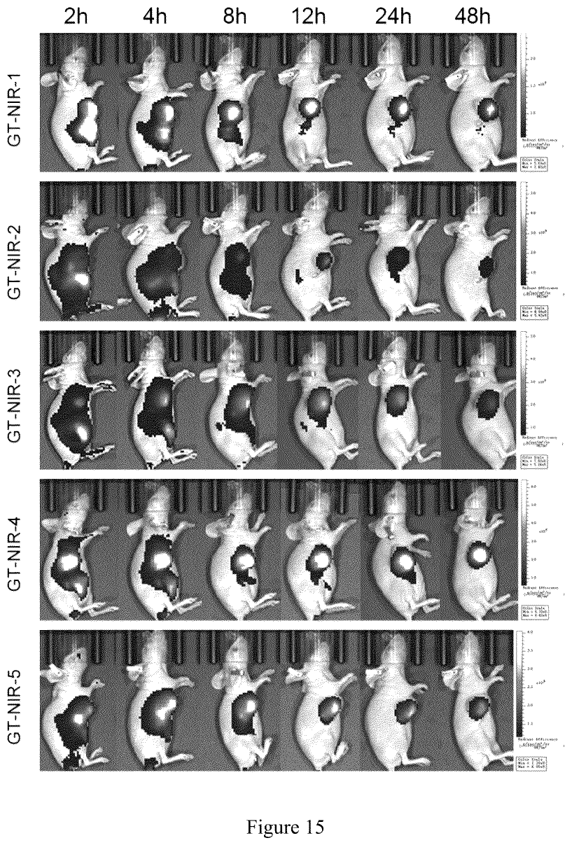

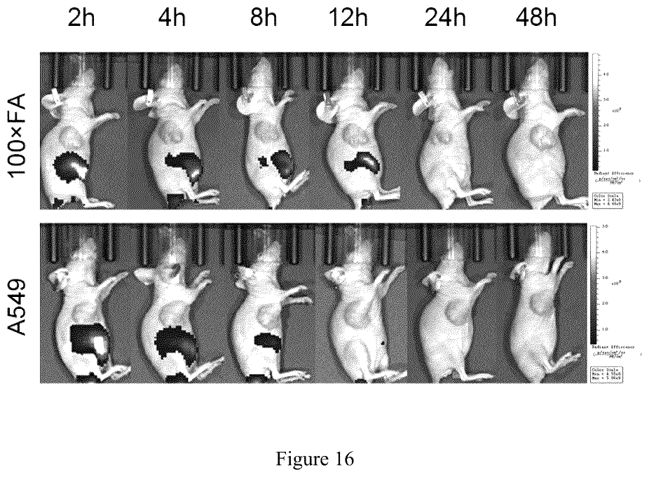

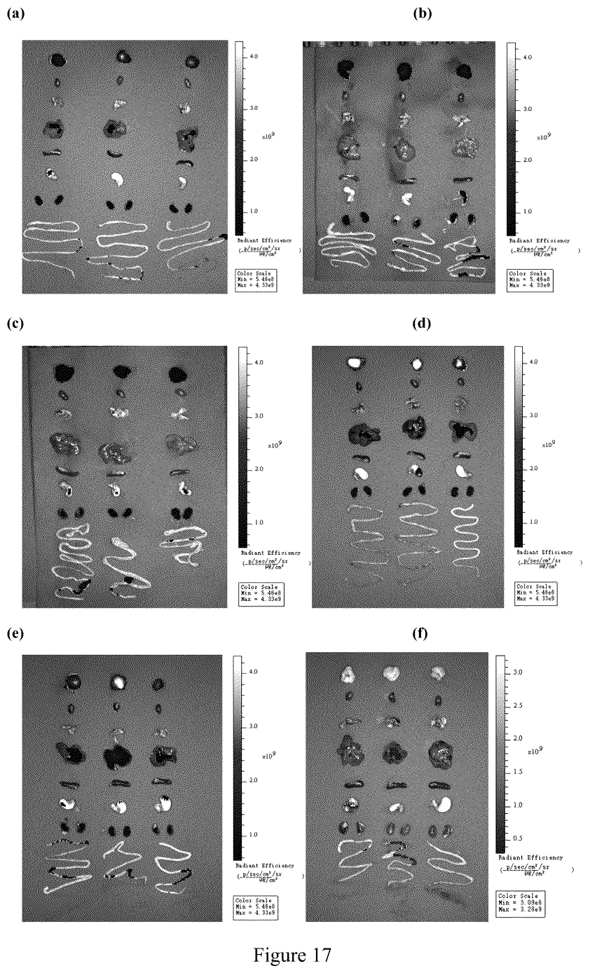

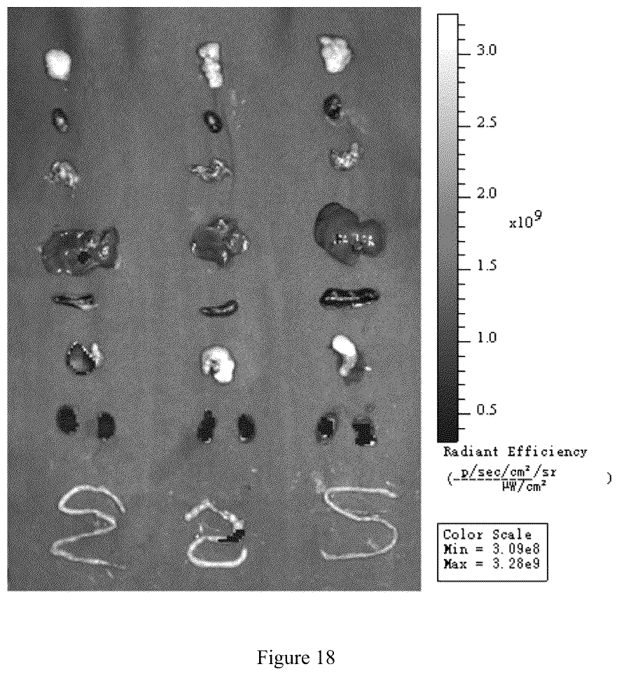

Whole-body fluorescence imaging was performed using an IVIS imaging system with 750-800 nm excitation light at 2, 4, 8, 12, 24, and 48 hours post-injection. At 8 hours after systemic imaging, the mice were euthanized, and organs were collected for ex vivo tissue biodistribution analysis using the IVIS system. The results demonstrated that the GT-NIR series compounds specifically targeted and enabled imaging of FR-positive KB xenografts in mice. In contrast, no targeted tumor imaging was observed with probe GT-NIR-4 in either the FR-negative A549 xenograft negative control group or the KB xenograft competition group co-administered with excess folic acid; instead, the probe was rapidly cleared from the body. These results are shown in FIG. 15 to FIG. 18.

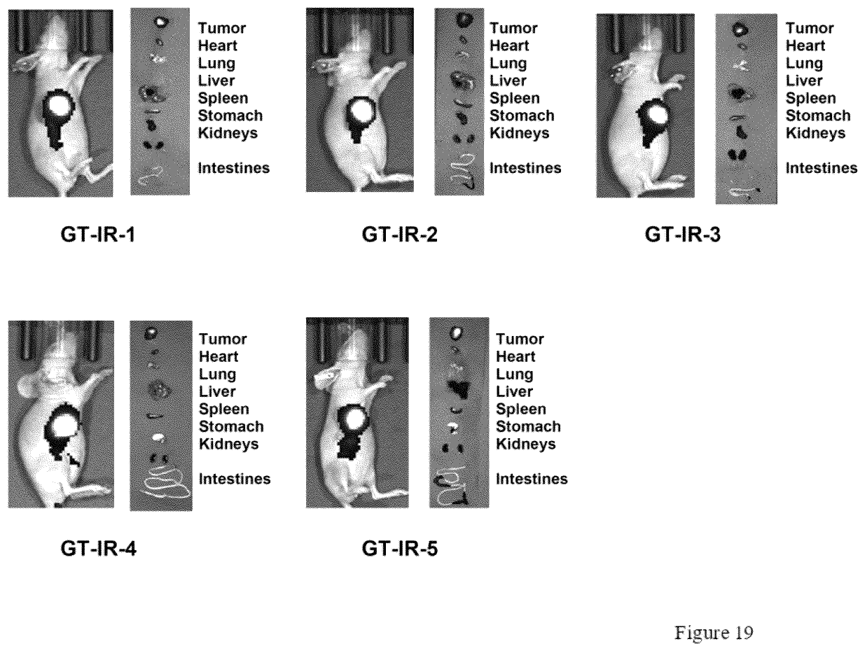

The in vivo imaging capability of the GT-IR series compounds was evaluated in the FR⁺ KB cell xenograft model following the same experimental procedure. Imaging was performed at 8 hours post-injection using the IVIS system with 750-800 nm excitation light. After euthanasia, organs were collected and subjected to ex vivo biodistribution analysis. FIG. 19 shows that GT-IR series compounds selectively accumulated at tumor sites, enabling tumor fluorescence imaging, indicating their suitability as tumor-targeted fluorescent probes for in vivo imaging.

### Example 49: Cytotoxicity Assessment of Fluorescent Probes

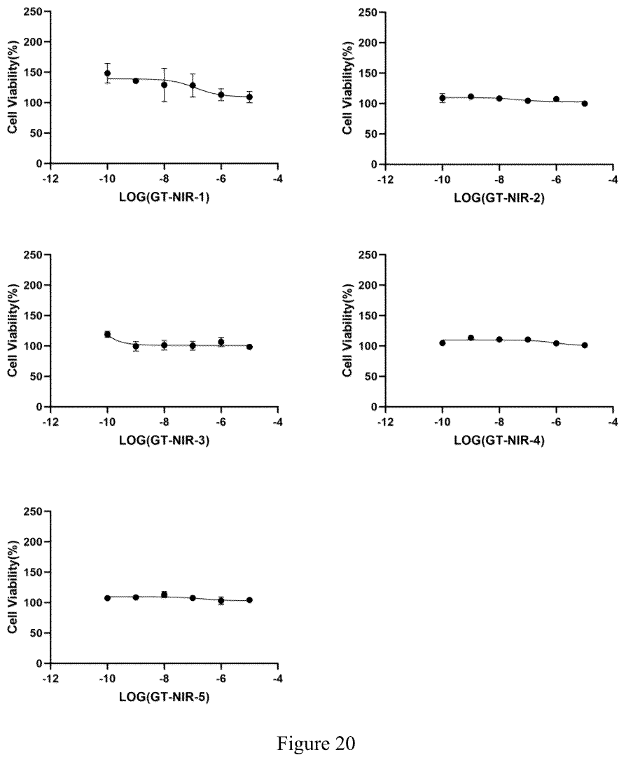

Since the targeting ligand (i.e., folate antagonist) possesses cytotoxic potential, the safety of the GT-NIR series probes was evaluated. HUVEC cells were routinely cultured in high-glucose DMEM medium supplemented with 10% FBS, 100 IU/mL penicillin, and streptomycin, and maintained at 37° C. in a humidified incubator with 5% CO₂. The cells were seeded in 96-well plates at a density of 4,000 cells per well and cultured overnight. The cells were then treated with media containing different concentrations of each probe from the GT-NIR series for 24 hours. Cell viability was assessed by adding 20 μL of Cell Counting Kit-8 reagent. Optical density was measured using a microplate reader with a 450 nm filter. The experimental results, calculated using GraphPad Prism software, represent the mean values from three independent experiments. The cytotoxicity results are shown in FIG. 20. All GT-NIR series probes exhibited minimal toxic effects on normal HUVEC cells.

While the present invention has been described in detail with reference to the foregoing embodiments, it will be understood by those skilled in the art that the technical solutions described in the embodiments may be modified, or some of the technical features may be equivalently substituted. Such modifications or substitutions do not depart from the spirit and scope of the technical solutions of the embodiments of the present invention.

What is claimed is:

1. A fluorescent probe compound for tumor-targeted imaging or a pharmaceutically acceptable salt thereof, characterized in that the compound has the structure represented by Formula I: 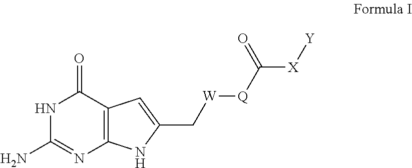
wherein W and Q are selected from the following structures:
(1) W is C(O)NH—, and Q is (CH₂)ⱼ—, wherein j is selected from 1, 2, 3, 4, or 5;
(2) W is (CH₂)ₙ— and Q is 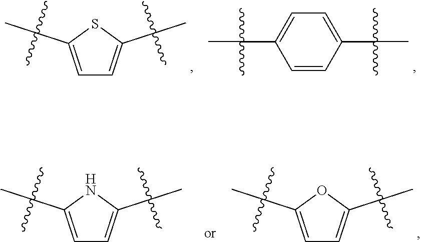
wherein n is selected from 1, 2, 3, 4, or 5;
X is an amino group or an amidation derivative group, wherein the amino group is independently selected from tyrosine, cysteine, glutamic acid, methionine, threonine, serine, 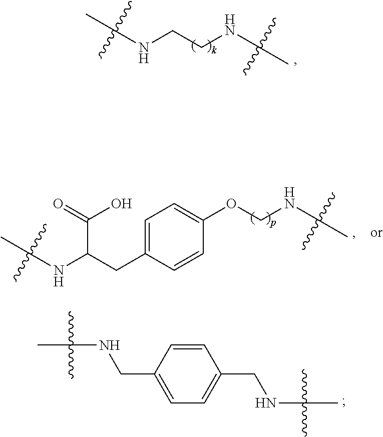
wherein the amidation derivative group represents an amidation derivative formed by condensation of two or more structures independently selected from said amino groups;
k is selected from 0, 1, 2, 3, 4, or 5;
p is selected from 1, 2, 3, or 4;
Y is independently selected from dyes having fluorescence excitation in the visible spectrum and emission in the near-infrared range, and the compound maintains or enhances the fluorescence of said dye.

2. The fluorescent probe compound for tumor-targeted imaging or a pharmaceutically acceptable salt thereof according to claim 1, wherein the amidation derivative group is selected from the group consisting of: 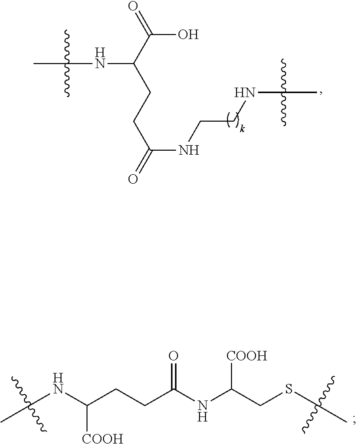
wherein k is selected from 0, 1, 2, 3, 4, or 5.

3. The fluorescent probe compound for tumor-targeted imaging or a pharmaceutically acceptable salt thereof according to claim 1, characterized in that Y is selected from the group consisting of FITC, dye IR-783, and S0456.

4. The fluorescent probe compound for tumor-targeted imaging or a pharmaceutically acceptable salt thereof according to claim 1, characterized in that the compound is selected from the group consisting of:

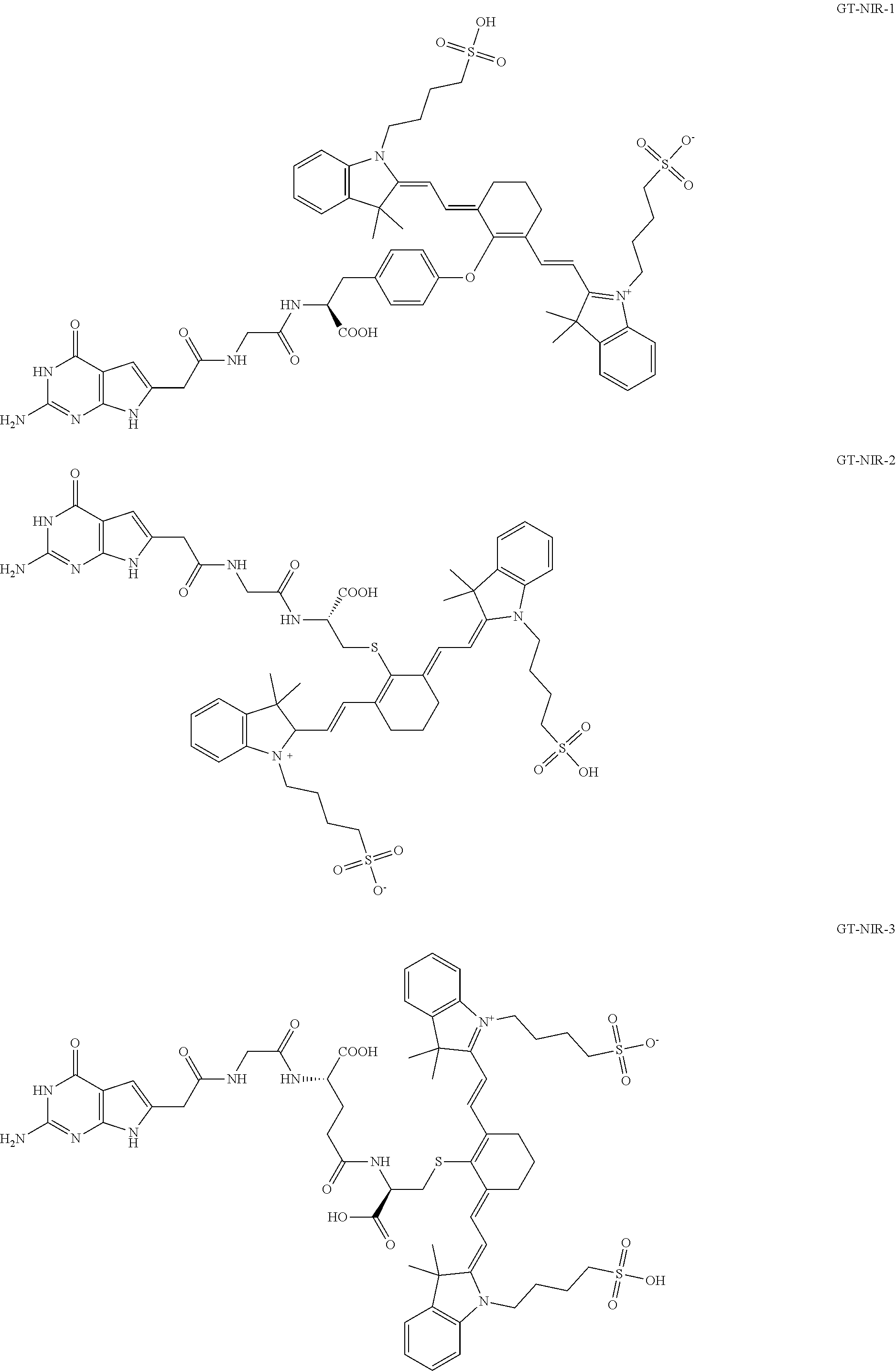 [SMILES: CC1(C)C(/C=C/C2=C(Oc3ccc(C[C@H](NC(=O)CNC(=O)Cc4cc5c(=O)[nH]c(N)nc5[nH]4)C(=O)O)cc3)C(=C/C=C3/N(CCCCS(=O)(=O)O)c4ccccc4C3(C)C)/CCC2)=[N+](CCCCS(=O)(=O)[O-])c2ccccc21 ; CC1(C)/C(=C\C=C2/CCCC(/C=C/C3N(CCCCS(=O)(=O)[O-])c4ccccc4C3(C)C)=C2SC[C@H](NC(=O)CNC(=O)Cc2cc3c(=O)[nH]c(N)nc3[nH]2)C(=O)O)N(CCCCS(=O)(=O)O)c2ccccc21 ; CC1(C)C(/C=C/C2=C(SC[C@H](NC(=O)CC[C@H](NC(=O)CNC(=O)Cc3cc4c(=O)[nH]c(N)nc4[nH]3)C(=O)O)C(=O)O)C(=C/C=C3/N(CCCCS(=O)(=O)O)c4ccccc4C3(C)C)/CCC2)=[N+](CCCCS(=O)(=O)[O-])c2ccccc21]

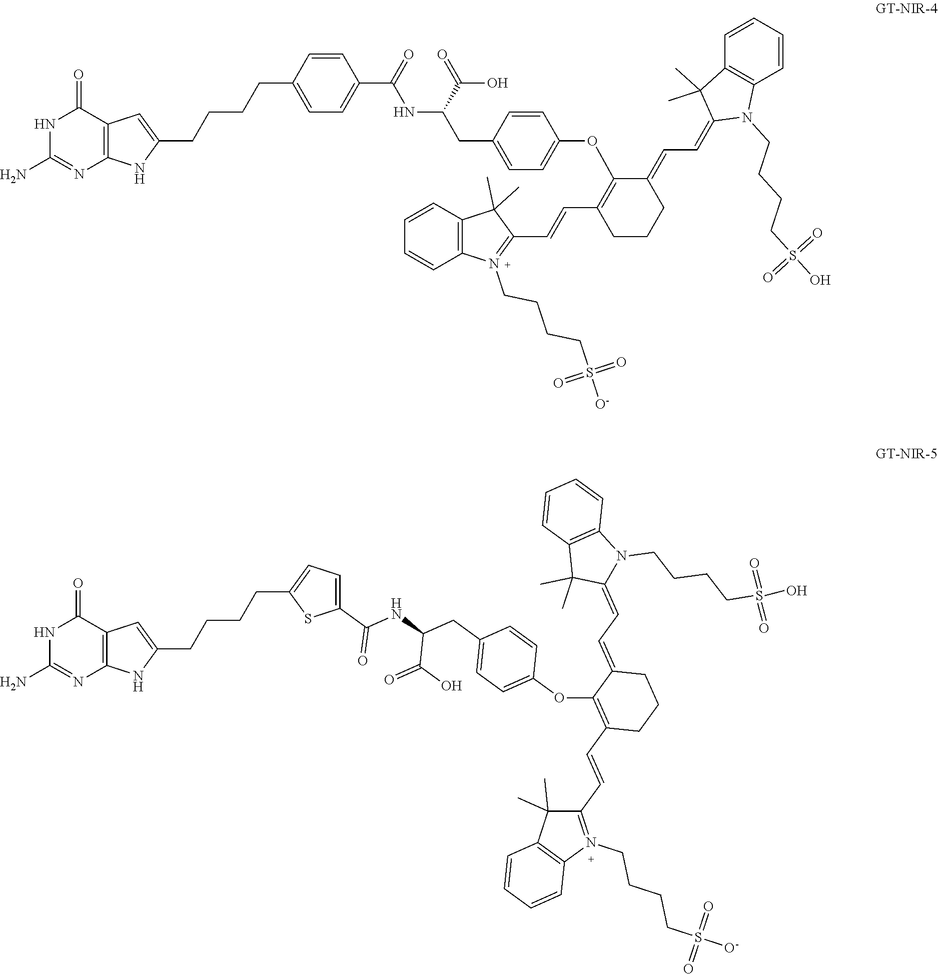

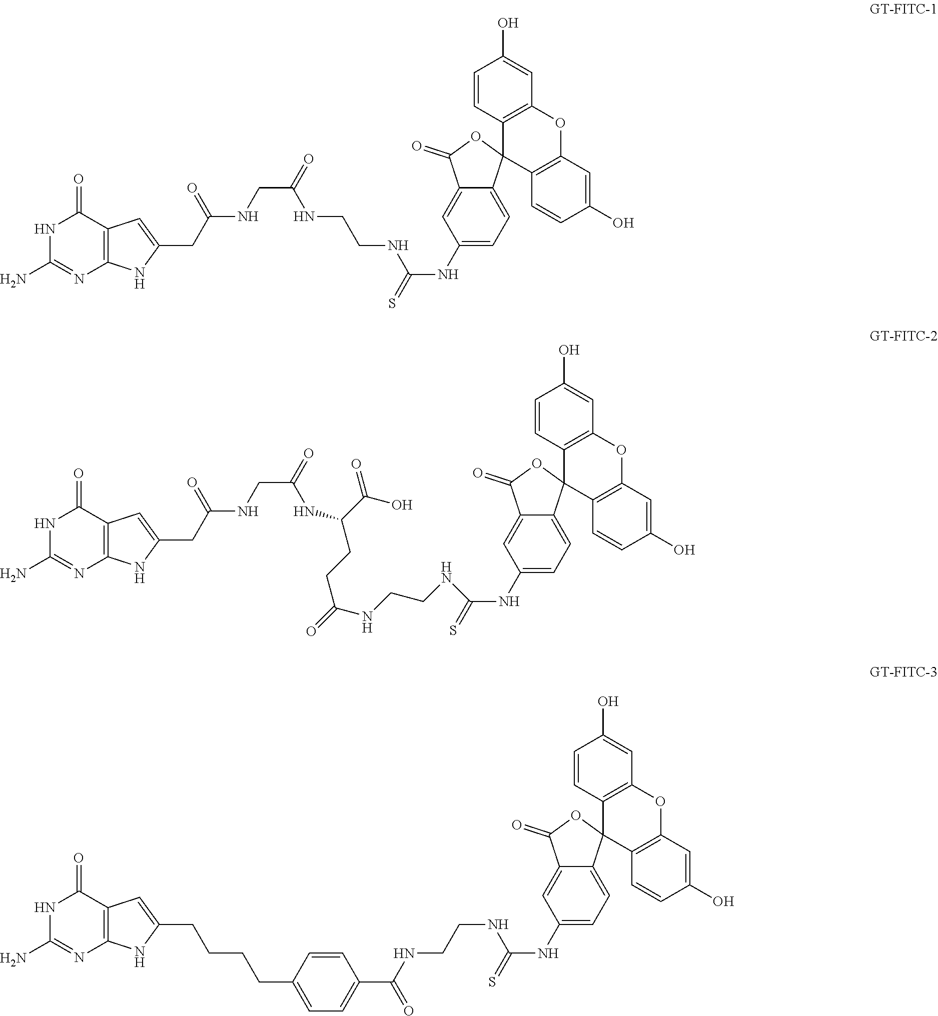 [SMILES: Nc1nc2[nH]c(CC(=O)NCC(=O)NCCNC(=S)Nc3ccc4c(c3)C(=O)OC43c4ccc(O)cc4Oc4cc(O)ccc43)cc2c(=O)[nH]1 ; Nc1nc2[nH]c(CC(=O)NCC(=O)N[C@@H](CCC(=O)NCCNC(=S)Nc3ccc4c(c3)C(=O)OC43c4ccc(O)cc4Oc4cc(O)ccc43)C(=O)O)cc2c(=O)[nH]1 ; Nc1nc2[nH]c(CCCCc3ccc(C(=O)NCCNC(=S)Nc4ccc5c(c4)C(=O)OC54c5ccc(O)cc5Oc5cc(O)ccc54)cc3)cc2c(=O)[nH]1]

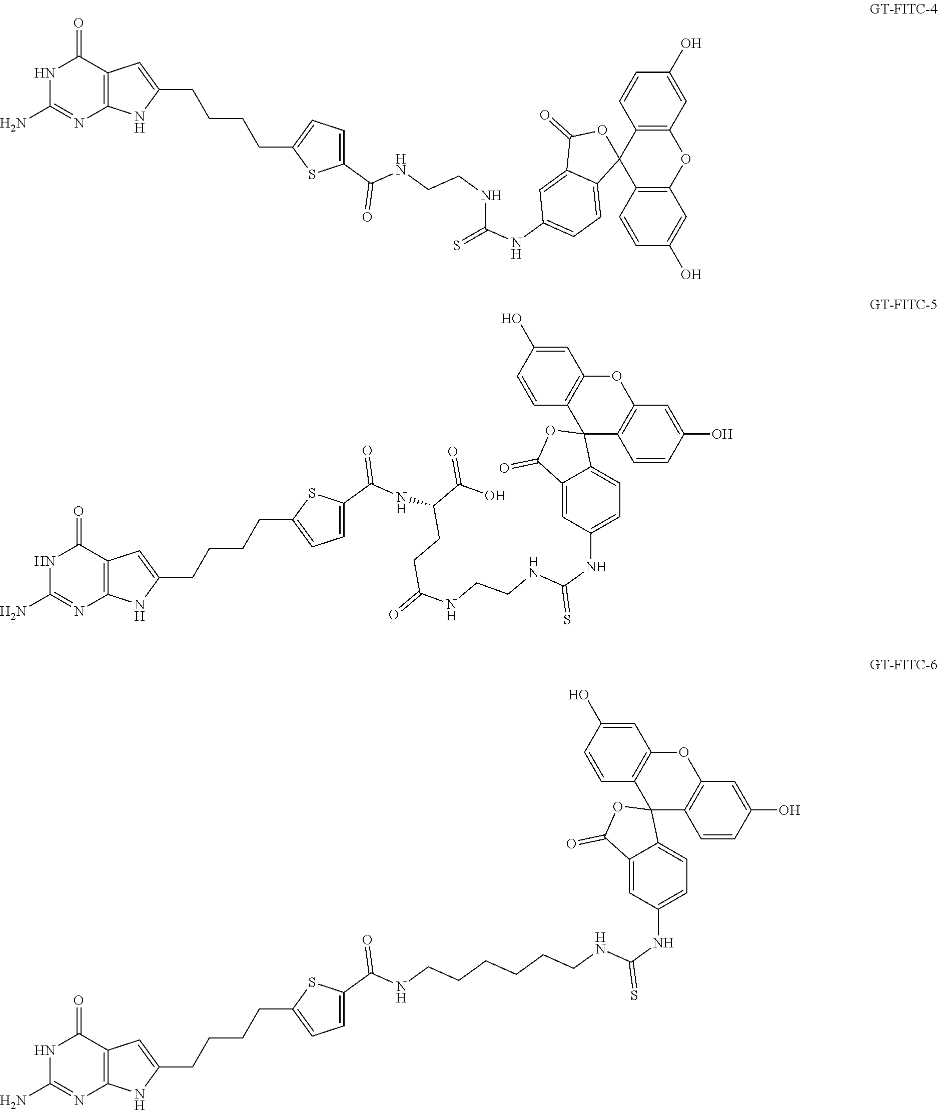 [SMILES: Nc1nc2[nH]c(CCCCc3ccc(C(=O)NCCNC(=S)Nc4ccc5c(c4)C(=O)OC54c5ccc(O)cc5Oc5cc(O)ccc54)s3)cc2c(=O)[nH]1 ; Nc1nc2[nH]c(CCCCc3ccc(C(=O)N[C@@H](CCC(=O)NCCNC(=S)Nc4ccc5c(c4)C(=O)OC54c5ccc(O)cc5Oc5cc(O)ccc54)C(=O)O)s3)cc2c(=O)[nH]1 ; Nc1nc2[nH]c(CCCCc3ccc(C(=O)NCCCCCCNC(=S)Nc4ccc5c(c4)C(=O)OC54c5ccc(O)cc5Oc5cc(O)ccc54)s3)cc2c(=O)[nH]1]

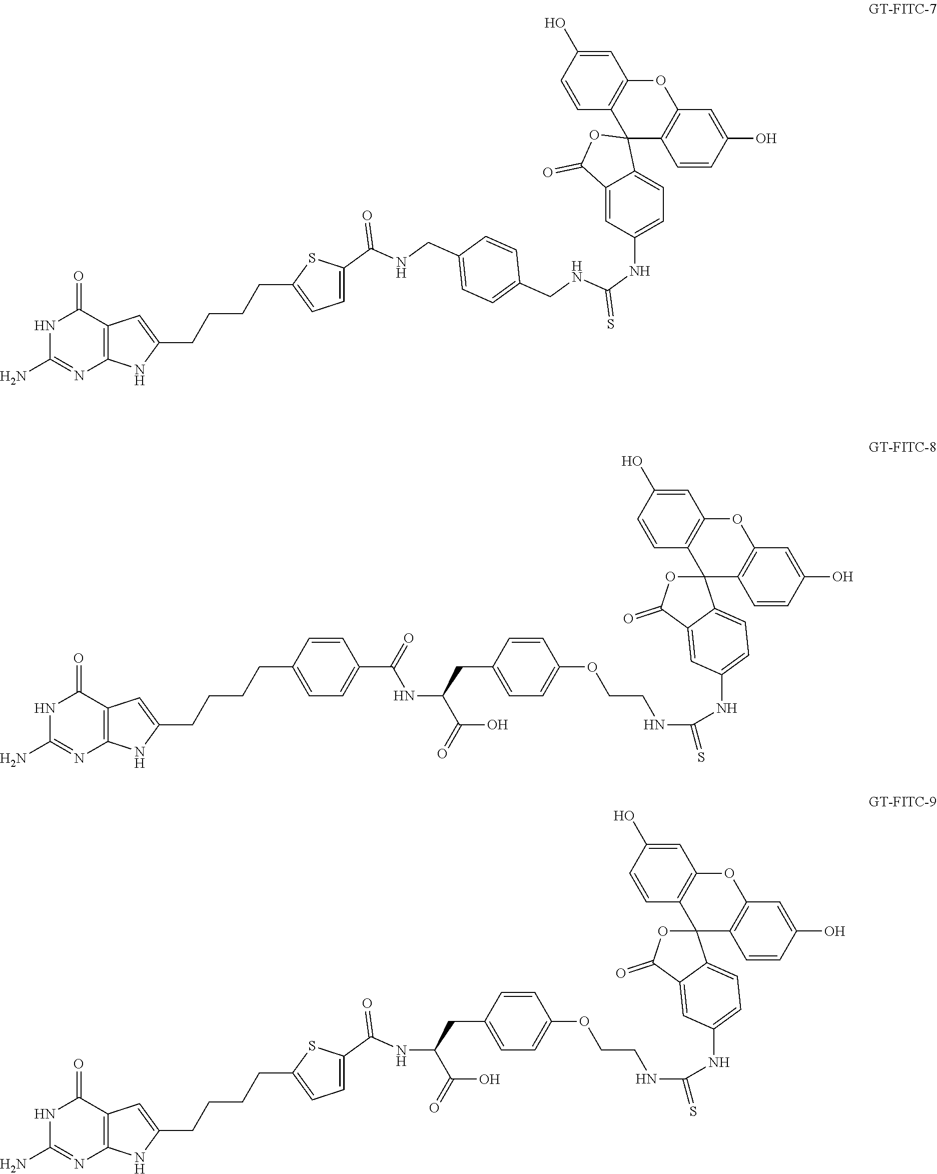 [SMILES: Nc1nc2[nH]c(CCCCc3ccc(C(=O)NCc4ccc(CNC(=S)Nc5ccc6c(c5)C(=O)OC65c6ccc(O)cc6Oc6cc(O)ccc65)cc4)s3)cc2c(=O)[nH]1 ; Nc1nc2[nH]c(CCCCc3ccc(C(=O)N[C@@H](Cc4ccc(OCCNC(=S)Nc5ccc6c(c5)C(=O)OC65c6ccc(O)cc6Oc6cc(O)ccc65)cc4)C(=O)O)cc3)cc2c(=O)[nH]1 ; Nc1nc2[nH]c(CCCCc3ccc(C(=O)N[C@@H](Cc4ccc(OCCNC(=S)Nc5ccc6c(c5)C(=O)OC65c6ccc(O)cc6Oc6cc(O)ccc65)cc4)C(=O)O)s3)cc2c(=O)[nH]1]

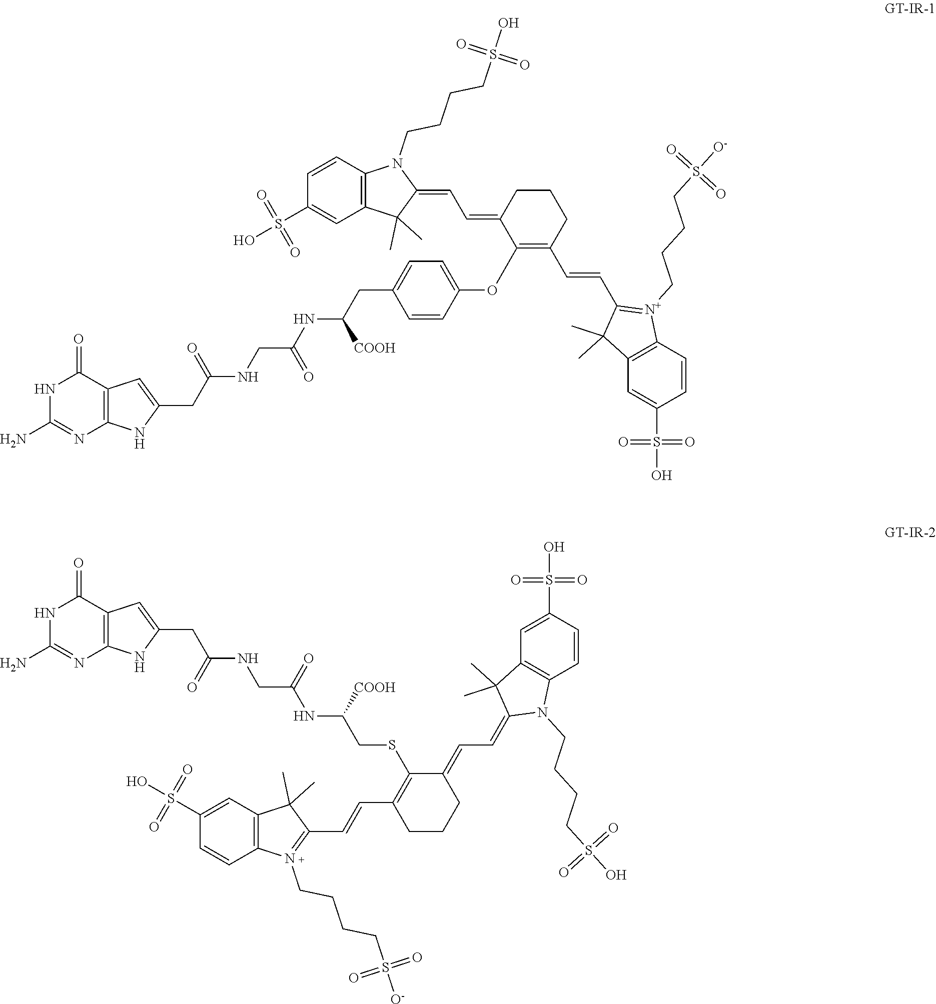

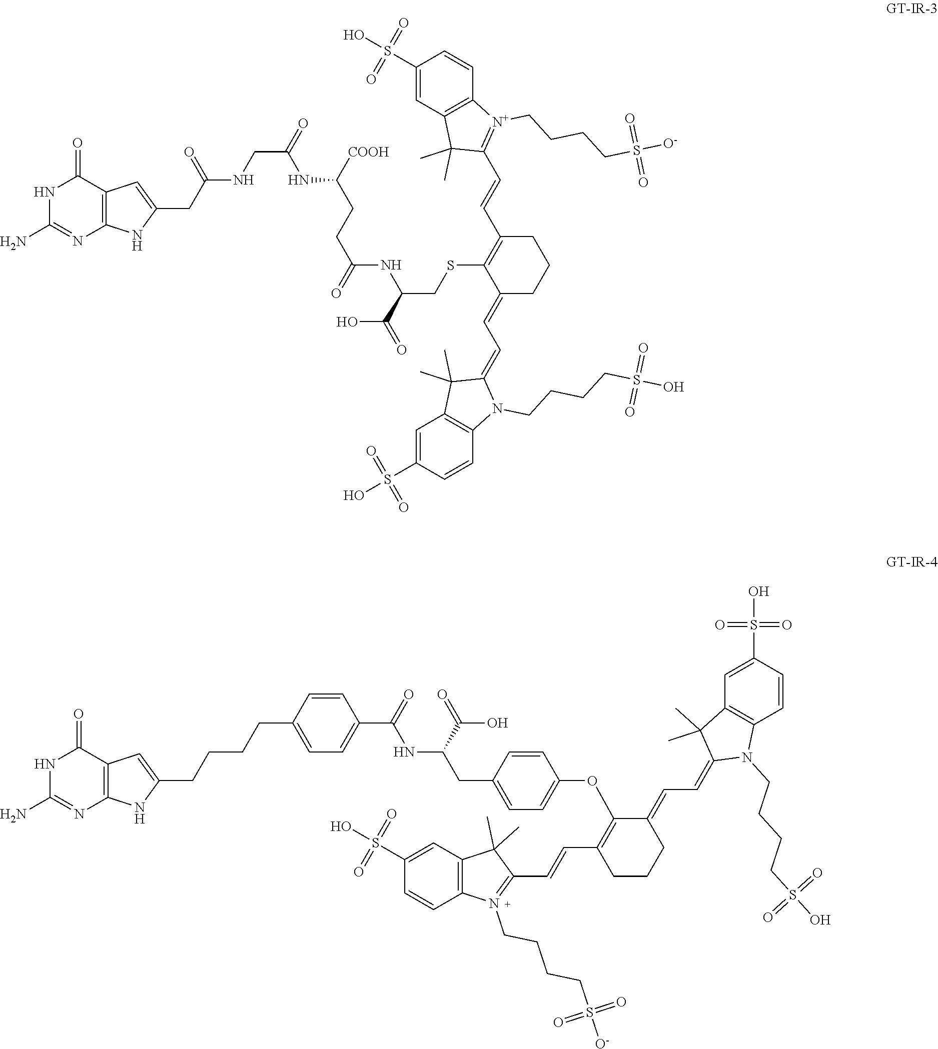

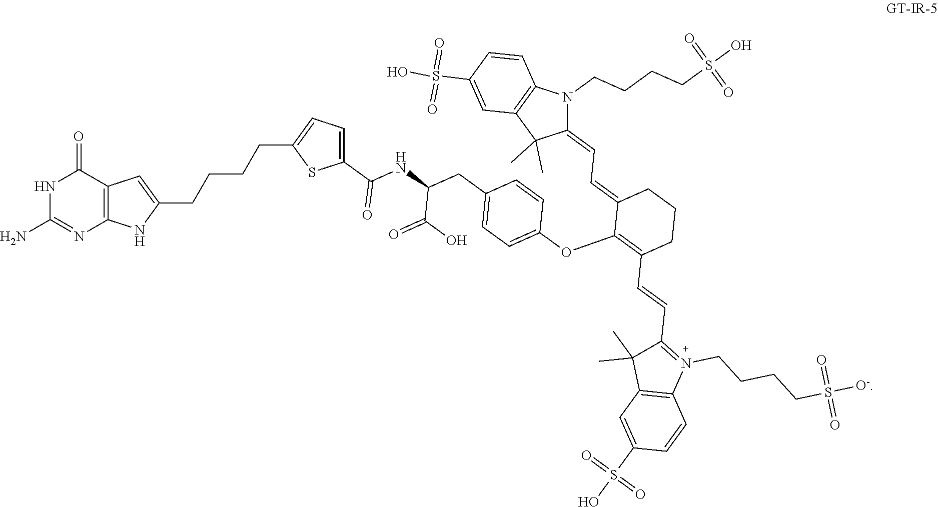

5. A composition comprising the fluorescent probe compound for tumor-targeted imaging according to claim 1, characterized in that it further comprises at least one pharmaceutically acceptable carrier or excipient.

6. Use of the fluorescent probe compound for tumor-targeted imaging according to claim 1 in the preparation of a tumor diagnostic reagent for tumor-targeted imaging.

7. The use according to claim 6, characterized in that the tumor is one or more selected from liver cancer, breast cancer, lung cancer, pancreatic cancer, ovarian cancer, and colorectal cancer.
